(19) United States
(12) Patent Application Publication
(10) Pub. No.: US 2006/0093554 A1
(43) Pub. Date: May 4, 2006

(54) Galenical formulations

(76) Inventors: Johannes Platzek, Berlin (DE); Ulrich Niedballa, Berlin (DE); Bernd Raduechel, Berlin (DE); Peter Mareski, Berlin (DE); Bernd Misselwitz, Glienicke (DE); Thomas Frenzel, Berlin (DE); Hanns-Joachim Weinmann, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/223,203

(22) Filed: Sep. 12, 2005

Related U.S. Application Data
(63) Continuation of application No. 09/672,049, filed on Sep. 29, 2000, now abandoned.
(60) Provisional application No. 60/158,302, filed on Oct. 8, 1999.

(30) Foreign Application Priority Data
Sep. 29, 1999 (DE) 199 48 651.4-43

(51) Int. Cl. A61K 51/00

(52) U.S. Cl. 424/1.69

(57) ABSTRACT

This invention describes new galenical formulations that contain paramagnetic and diamagnetic perfluoroalkyl-containing compounds. The new formulations are suitable as contrast media for nuclear spin tomography.

The invention relates to the field of galenical formulations, which are used in particular as contrast media for the visualization of lymph nodes. The invention relates to the subject that is characterized in the claims, namely new formulations that contain paramagnetic and diamagnetic perfluoroalkyl-containing substances.

Malignant tumors metastasize heaped in regional lymph nodes, whereby several lymph node stations can also be involved. Thus, lymph node metastases are found in about 50-69% of all patients with malignant tumors (Elke, Lymphographie [Lymphography], in: Frommhold, Stender, Thurn (eds.), Radiologische Diaghostik in Klinik und Praxis [Radiological Diagnosis in Clinical Studies and Practice], Volume IV, Thieme Verlag Stuttgart, 7th Ed., 434-496, 1984). The diagnosis of a metastatic attack of lymph nodes is of great importance with respect to the therapy and prognosis of malignant diseases. With the modern imaging methods (CT, US, and MRT), lymphogenous metastasis sites of malignant tumors are detected only inadequately, since in most cases, only the size and the shape of the lymph node can be used as a diagnostic criterion. Thus, small metastases in non-enlarged lymph nodes (<2 cm) are not distinguished from lymph node hyperplasias without a malignant attack (Steinkamp et al., Sonographie und Kernspintomographie: Differentialdiagnostik von reaktiver Lymphknotenvergröberung und Lymphknotenmetastasen am Hals [Sonography and Nuclear Spin Tomography: Differential Diagnosis of Reactive Lymph Node Enlargement and Lymph Node Metastases on the Neck], Radiol. Diagn. 33: 158, 1992).

It would be desirable to distinguish between-lymph nodes with metastatic attack and hyperplastic lymph nodes with the aid of specific contrast media. In this case, the contrast medium could be administered intravasally or interstitially/intracutaneously (see above Siefert, H. M. et al., Lymphology 13, 150-157, 1980). The interstitial/intracutaneous administration has the advantage that the substance is transported directly from the scattering focus (e.g., primary tumor) by the corresponding lymph tract into the potentially related regional lymph node stations. Likewise, a high concentration of the contrast medium in the lymph nodes can be achieved with a low dose. Such markers that are to be administered interstitially are mainly used to date in the; nuclear-medicine evaluation (with use of radioactive particles, such as, e.g., ¹⁹⁸Au-colloid). Nuclear-medicine methods have only a very inadequate spatial resolution, however, in contrast to nuclear spin tomography with its high spatial resolution, which lies in the range of fractions of a millimeter. The direct x-ray-lymphography (injection of an oily contrast medium suspension in a prepared lymph vessel) is an invasive method that is used only very rarely and that can visualize only a few lymph outflow stations. Fluorescence-labeled dextrans are also used experimentally in animal experiments to be able to observe the lymph outflow after their interstitial administration. All commonly used markers for the visualization of lymph tracts and lymph nodes after interstitial/intracutaneous administration have in common the fact that they are substances with a particulate nature (“particulates,” e.g., emulsions and nanocrystal suspensions) or large polymers (see above, WO 90/14846). The previously described preparations have proven to be of value, however, based on their inadequate local and systemic compatibility as well as their small lymph passageway, which produces an inadequate diagnostic efficiency, in most cases unsuitable for indirect lymphography.

There is generally a great need, therefore, for a lymph-specific MRT-contrast medium with suitable pharmaceutical and pharmacological properties. In the case of the pharmaceutical properties, focus is placed first on the highest possible contrast medium concentration and an adequate stability. In the case of the pharmacological properties, and in addition to a diagnostically relevant lymph concentration that is as uniform as possible over several (or in the case of intravenous administration over all) lymph stations, focus is placed mainly on a quick and complete excretion of the contrast medium to avoid an unnecessary load of the entire organism. Moreover, corresponding preparations must have at their disposal an adequate local and acute compatibility.

With respect to the application in radiological practice and in addition to as simple an application as possible of corresponding preparations, the quick “start-up” of the preparations is of central importance. Thus, if at all possible, it should be possible to perform imaging within a few hours after the administration of the contrast media.

Contrast media that are suitable for the visualization of lymph nodes are already described in German Laid-Open Specification DE 196 03 033. There, perfluoroalkyl-containing metal complexes are disclosed, which are preferably used as lymphographic agents (see FIG. 1 of DE 196 03 033). Similar metal complexes that are suitable especially as blood-pool agents are described in German Laid-Open Specification DE 197 29 013. These compounds are already quite well suited as contrast media in lymphography, however, it is desirable to further improve the pharmaceutical and pharmacological properties of a contrast medium formulation.

The object of the invention is therefore to make available new galenical formulations that are suitable as contrast media especially for the visualization of lymph nodes and that meet the above-mentioned pharmaceutical and pharmacological requirements.

This object is achieved by the galenical formulations of this invention.

The galenical formulations of this invention contain paramagnetic perfluoroalkyl-containing compounds, which were already described in, e.g., laid-open specifications DE 196 03 033, DE 197 29 013, and WO 97/26017, and in addition diamagnetic perfluoroalkyl-containing substances. The paramagnetic perfluoroalkyl-containing compounds are compounds of general formula I
R^{F}-A (I)

in which R^{F} represents a straight-chain or branched perfluoroalkyl radical with 4 to 30 carbon atoms, and A is a molecule portion that contains 1-6 metal complexes. Molecule portion A stands for, for example, a group L-M, whereby L stands for a linker and M stands for a metal complex that consists of an open-chain or cyclic chelating agent, which contains an atom of atomic numbers 21-29, 39, 42, 44 or 57-83 as a central atom. In this case, linker L is a direct bond, a methylene group, an —NHCO group, a group 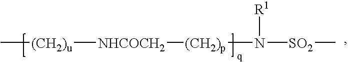
whereby p means the numbers 0 to 10, q and u, independently of one another, mean the numbers 0 or 1, and
R¹ means a hydrogen atom, a methyl group, a —CH₂—OH group, a —CH₂—CO₂H group or a C₂-C₁₅ chain, which optionally is interrupted by 1 to 3 oxygen atoms, 1 to 2 >CO-groups or an optionally substituted aryl group and/or is substituted with 1 to 4 hydroxyl groups, 1 to 2 C₁-C₄ alkoxy groups, 1 to 2 carboxy groups,
or a straight-chain, branched, saturated or unsaturated C₂-C₃₀ carbon chain, which optionally contains 1 to 10 oxygen atoms, 1 to 3 —NR¹ groups, 1 to 2 sulfur atoms, a piperazine, a —CONR¹ group, an —NR¹CO group, an —SO₂ group, an —NR¹—CO₂ group, 1 to 2 CO groups, a group 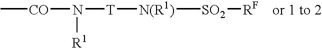
optionally substituted aryls and/or is interrupted by these groups and/or is optionally substituted with 1 to 3 —OR¹ groups, 1 to 2 oxo groups, 1 to 2 —NH—COR¹ groups, 1 to 2 —CONHR¹ groups, 1 to 2 (—CH₂)ₚ—CO₂H groups, 1 to 2 groups —(CH₂)ₚ—(O)_{q}CH₂CH₂R^{F},
whereby
R¹₁ R^{F} and p and q have the above-indicated meanings, and
T means a C₂-C₁₀ chain, which optionally is interrupted by 1 to 2 oxygen atoms or 1 to 2 —NHCO groups.

In this case, metal complex M stands for the following metal complexes:
a complex of general formula II 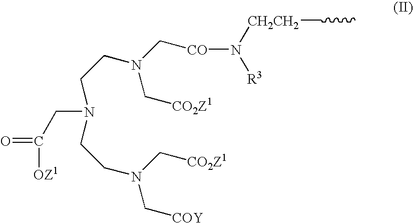
in which R³, Z¹ and Y are independent of one another, and
R³ has the meaning of R¹ or —(CH₂)ₘ-L-R^{F}, whereby m is 0, 1 or 2, and L and RF have the above-mentioned meaning,
Z¹, independently of one another, mean a hydrogen atom or a metal ion equivalent of atomic numbers 21-29, 39, 42, 44 or 57-83,
Y means —OZ¹, or 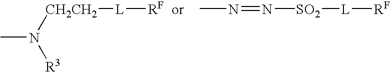
whereby Z¹, L, R^{F} and R³ have the above-mentioned meanings,
a complex of general formula III 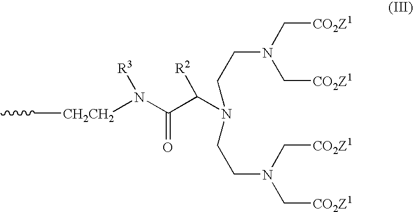
in which R³ and Z¹ have the above-mentioned meanings, and R² has the meaning of R¹,
a complex of general formula IV 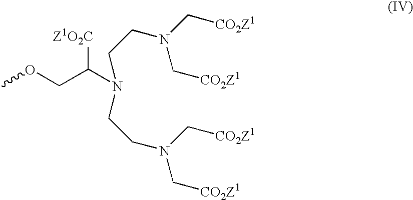
in which Z¹ has-the above-mentioned meaning,
a complex of general formula V 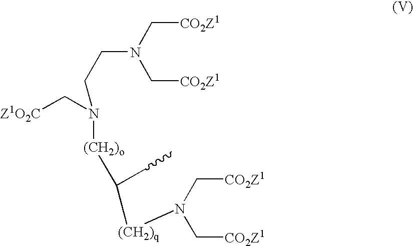
in which Z¹ has the above-mentioned meaning, and o and q stand for the numbers 0 or 1, and yields the sum o+q=1,
a complex of general formula VI 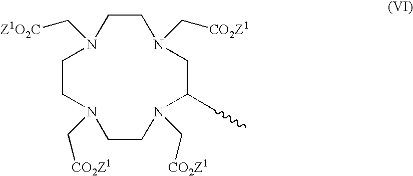
in which Z¹ has the above-mentioned meaning,
a complex of general formula VII 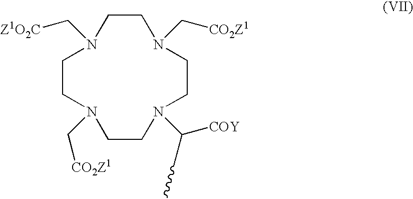
in which Z¹ and Y have the above-mentioned meanings,
a complex of general formula VIII 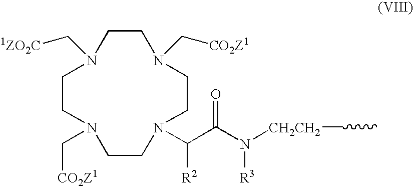
in which R³ and Z¹ have the above-mentioned meanings, and R² has the above-mentioned meaning of R¹,
a complex of general formula IX 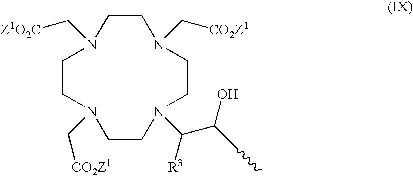
in which R³ and Z¹ have the above-mentioned meanings,
a complex of general formula X 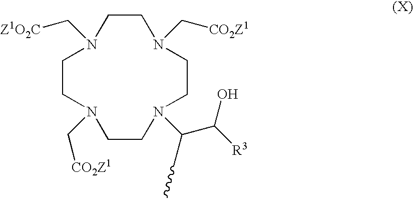
in which R³ and Z¹ have the above-mentioned meanings,
a complex of general formula XI 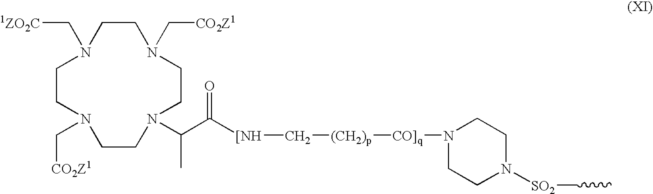
in which Z¹₁ p and q have the above-mentioned meaning, and R² has the meaning of R¹,
a complex of general formula XII 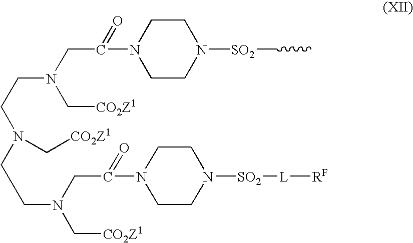
in which L, RF and Z¹ have the above-mentioned meanings,
a complex of general formula XIII 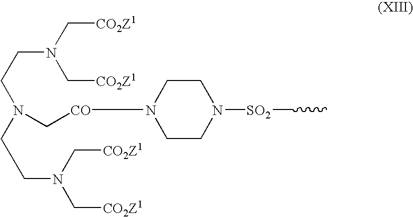
in which Z¹ has the above-mentioned meaning.

Such compounds and production thereof have been described in German Laid-Open Specification DE 196 03 033 A1 and in International Patent Application WO 97/26017.

Molecule portion A according to formula I can also exhibit the following structure: 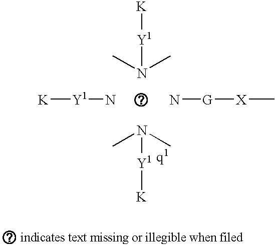
whereby
q¹ is a number 0, 1, 2 or 3,
K stands for a complexing agent or metal complex or salts thereof of organic and/or inorganic bases or amino acids or amino acid amides,
X is a direct bond for the perfluoroalkyl group, a phenylene group or a C₁-C₁₀ alkyl chain, which optionally contains 1-15 oxygen atoms, 1-5 sulfur atoms, 1-10 carbonyl groups, 1-10 (NR) groups, 1-2 NRSO₂ groups, 1-10 CONR groups, 1 piperidine group, 1-3 SO₂ groups, 1-2 phenylene groups or optionally is substituted by 1-3 radicals R^{F}, in which R stands for a hydrogen atom, a phenyl, benzyl or a C₁-C₁₅ alkyl group, which optionally contains 1-2 NHCO groups, 1-2 CO groups, 1-5 oxygen atoms and optionally is substituted by 1-5 hydroxy, 1-5 methoxy, 1-3 carboxy, 1-3 R^{F} radicals,
Y¹ is a direct bond or a chain of general formula II′ or III′: 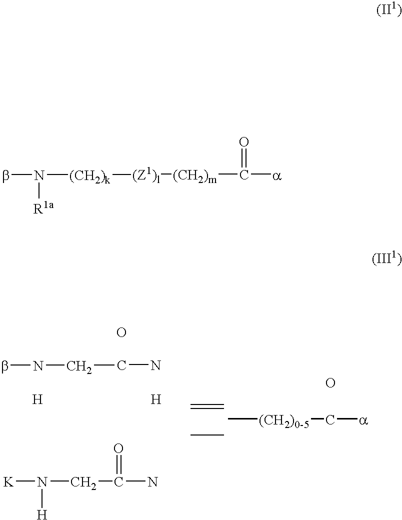
in which
R^{1a} is a hydrogen atom, a phenyl group, a benzyl group or a C₁-C₇ alkyl group, which optionally is substituted with a carboxy group, a methoxy group or a hydroxy group,
Z¹ is a direct bond, a polyglycol ether group with up to 5 glycol units or a molecule portion of general formula IV¹
   —CH(R^{2a})— (IV¹)
in which R^{2a} is a C₁-C₇ carboxylic acid, a phenyl group, a benzyl group or a —(CH₂)₁₋₅—NH—K group,
α represents the binding to the nitrogen atom of the skeleton chain, β represents the binding to the complexing agent or metal complex K,
and in which variables k and m stand for natural
   numbers between 0 and 10, and 1 stands for 0 or 1, and whereby
G is a CO or SO₂ group.

Such compounds and the production thereof are described in German Laid-Open Specification DE 197 29 013 A1.

Molecule portion A according to general formula I can also stand for a group L¹-M¹, in which L¹ stands for a linker and M¹ stands for a metal complex. In this case, linker L¹ is a molecule portion according to general formula XIV 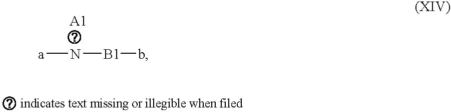
in which
N represents a nitrogen atom,
A1 means a hydrogen atom, a straight-chain or branched C₁-C₃₀ alkyl group, which optionally is interrupted by 1-15 oxygen atoms and/or optionally is substituted with 1-10 hydroxy groups, 1-2 COOH groups, a phenyl group, a benzyl group and/or 1-5 —OR⁴ groups, with R⁴ in the meaning of a hydrogen atom or a C₁-C₇ alkyl radical, or B1-R^{F},
B1 means a straight-chain or branched C₁-C₃₀ alkylene group that optionally is interrupted by 1-10 oxygen atoms, 1-5 —NH-CO groups, 1-5 —CO—NH groups, by a phenylene group (that is optionally substituted by a COOH group), 1-3 sulfur atoms, 1-2 —N(B2)-SO₂ groups, and/or 1-2 —SO₂—N(B2) groups with B2 in the meaning of Al, an NHCO group, a CONH group, an N(B2)-SO₂ group, or an —SO₂—N(B2) group and/or optionally is substituted with radical R^{F}, and in which a represents the binding to metal complex M, and b
represents the binding to perfluoroalkyl group R^{F}.

In this case, metal complex M¹ stands for a metal complex of general formula XV 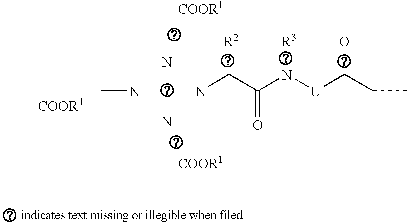
whereby R¹ stands for a hydrogen atom or a metal ion equivalent of atomic numbers 21-29, 31, 32, 37-39, 42-44, 49 or 57-83,
   R² and R³ stand for a hydrogen atom, a C₁-C₇ alkyl group, a benzyl group, a phenyl group, —CH₂OH or —CH₂—OCH₃,
   U stands for radical L, whereby L and U, independently of one another, can be the same or different, however.

Such compounds and the production thereof are described in the German patent application with the file number 199 14 101.0 as well as in the examples below.

Especially preferred are metal complexes in which the central atom is a gadolinium atom (atomic number 64). Metal complexes with cyclic chelating agents are preferred compared to those with open-chain chelating agents.

Especially preferred gadolinium complexes are the gadolinium complex of 10-[1-methyl-2-oxo-3-aza-5-oxo-5-{4-perfluorooctylsulfonyl-piperazin-1-yl}-pentyl]-1,4,7-tris(carboxymethyl)-1,4,7,10-tetraazacyclododecane (for production, see WO 97/26017, Example 33),

the gadolinium complex of 10-[2-hydroxy-4-aza-5-oxo-7-oxa-10,10,11,11,12,12,13,13,14,14,15,15,16,16,17,17,17-heptadecafluoroheptadecyl]-1,4,7-tris(carboxymethyl)-1,4,7,10-tetraazacyclododecane (for production, see DE 196 03 033, Example 2),

1,4,7-tris{1,4,7-tris(N-carboxylatomethyl)-10-(N-1-methyl-3,6-diaza-2,5,8-trioxooctane-1,8-diyl)-1,4,7,10-tetraazacyclododecane, Gd-complex}-10-(N-2H,2H,4H,4H,5H,5H-3-oxa-perfluoro-tridecanoyl)-1,4,7,10-tetraazacyclododecane (for production, see DE 197 29 013, Example 1),

1,4,7-tris{1,4,7-tris[(N-carboxylatomethyl)]-10-[N-1-methyl-3-aza-2,5-dioxopentane-1,5-diyl]-1,4,7,10-tetraazacyclododecane, Gd complex}-10-[2-(N-ethyl-N-perfluorooctylsulfonyl)-amino]-acetyl-1,4,7,10-tetraazacyclododecane (for production, see DE 197 29 013, Example 12),

the gadolinium complex of 10-[2-hydroxy-4-aza-5-oxo-7-aza-7-(perfluorooctylsulfonyl)-nonyl]-1,4,7-tris(carboxymethyl)-1,4,7,10-tetraazacyclododecane (for production, see DE 196 03 033, Example 1),

1,4,7-tris(carboxylatomethyl)-10-((3-aza-4-oxo-hexan-5-ylic)-acid-N-(2,3-dihydroxy-propyl)-N-(1H,1H,2H,2H,4H,4H,5H,5H-3-oxa)-perfluorotridecyl)-amide]-1,4,7,10-tetraazacyclododecane, gadolinium complex (for production see examples),

1,4,7-tris(carboxylatomethyl)-10-[(3-aza-4-oxo-hexan-5-ylic)-acid-N-(1H,1H,2H,2H,4H,4H,5H,5H-3-oxa-perfluorotridecyl)-amide]-1,4,7,10-tetraazacyclododecane, gadolinium complex (for production see examples),

1,4,7-tris(carboxylatomethyl)-10-{(3-aza-4-oxo-hexan-5-ylic)-acid-[N-3,6,9,12,15-pentaoxa)-hexadexyl)-N-(1H,1H,2H,2H,4H,4H,5H,5H-3-oxa)-perfluorotridecyl]-amide}-1,4,7,10-tetraazacyclododecane, gadolinium complex (for production, see examples),

and 1,4,7-tris(carboxylatomethyl)-10-[(3-aza-4-oxo-hexan-5-ylic)-acid-N-(5-hydroxy-3-oxa-pentyl)-N-(1H,1H,2H,2H,4H,4H,5H,5H-3-oxa)-perfluorotridecyl)-amide]-1,4,7,10-tetraazacyclododecane, gadolinium complex (for production, see examples).

The diamagnetic perfluoroalkyl-containing substances are those of general formula XVI:
R^{F}-L²-B² XVI
in which R^{F} represents a straight-chain or branched perfluoroalkyl radical with 4 to 30 carbon atoms, L² stands for a linker and B² stands for a hydrophilic group. Linker L² is a direct bond, an —SO₂ group or a straight-chain or branched carbon chain with up to 20 carbon atoms, which can be substituted with one or more —OH, —COO—, —SO₃ groups and/or optionally contains one or more —O—, —S—, —CO—, —CONH—, —NHCO—, —CONR—, —NRCO—, —SO₂—, —PO₄′—, —NH, —NR groups, an aryl ring or a piperazine, whereby R stands for a C₁ to C₂₀ alkyl radical, which in turn can contain one or more O atoms and/or can be substituted with —COO⁻ or SO₃ groups.

The hydrophilic group B² is a monosaccharide or a disaccharide, one or more adjacent —COO⁻ or −SO₃⁻ groups, a dicarboxylic acid, an isophthalic acid, a picolinic acid, a benzenesulfonic acid, a tetrahydropyran dicarboxylic acid, a 2,6-pyridinedicarboxylic acid, a quaternary ammonium ion, an aminopolycarboxylic acid, an aminodipolyethyleneglycoisulfonic acid, an aminopolyethylene glycol group, an SO₂—(CH₂)₂—OH group, a polyhydroxyalkyl chain with at least two hydroxyl groups or one or more polyethylene glycol chains with at least two glycol units, whereby the polyethylene glycol chains are terminated by an —OH or —OCH₃ group. Such substances are partially already known, and those substances for the production of the formulations according to the invention were newly synthesized. Known perfluoroalkyl-containing substances and the production thereof are described in the following publications:

J. G. Riess, Journal of Drug Targeting, 1994, Vol. 2, pp. 455-468;

J. B. Nivet et al., Eur. J. Med. Chem., 1991, Vol. 26, pp. 953-960;

M.-P. Krafft et al., Angew. Chem. [Applied Chemistry], 1994, Vol. 106, No. 10, pp. 1146-1148;

M. Lanier et al., Tetrahedron Letters, 1995, Vol. 36, No. 14, pp. 2491-2492;

F. Guillod et al., Carbohydrate Research, 1994, Vol. 261, pp. 37-55;

S. Achilefu et al., Journal of Fluorine Chemistry, 1995, Vol. 70, pp. 19-26;

L. Clary et al., Tetrahedron, 1995, Vol. 51, No. 47, pp. 13073-13088;

F. Szoni, et al., Journal of Fluorine Chemistry, 1989, Vol. 42, pp. 59-68;

H. Wu et al.;, Supramolecular Chemistry, 1994, Vol. 3, pp. 175-180;

F. Guileri et al., Angew. Chem. 1994, Vol. 106, No. 14, pp. 1583-1585;

M.-P. Krafft et al., Eur. J. Med. Chem., 1991, Vol. 26, pp. 545-550;

J. Greiner et al., Journal of Fluorine Chemistry, 1992, Vol. 56, pp. 285-293;

A. Milius et al., Carbohydrate Research, 1992, Vol. 229, pp. 323-336;

J. Riess et al., Colloids and Surfaces A, 1994, Vol. 84, pp. 33-48;

G. Merhi et al., J. Med. Chem., 1996, Vol. 39, pp. 4483-4488;

V. Cirkva et al., Journal of Fluorine Chemistry, 1997, Vol. 83, pp. 151-158;

A. Ould Amanetoullah et al., Journal of Fluorine Chemistry, 1997, Vol. 84,. pp. 149-153;

J. Chen et al., Inorg. Chem., 1996, Vol. 35 pp. 1590-161;

L. Clary et al., Tetrahedron Letters, 1995, Vol. 36, No. 4, pp. 539-542;

M. M. Chaabouni et al., Journal of Fluorine Chemistry, 1990, Vol. 46, pp. 307-315;

A. Milius et al., New J. Chem., 1991, Vol. 15, pp. 337-344;

M.-P. Krafft et al., New J. Chem., 1990, Vol. 14, pp. 869-875;

J.-B. Nivet et al., New J. Chem., 1994, Vol. 18, pp. 861-869;

C. Santaella et al., New J. Chem., 1991, Vol. 15, pp. 685-692;

C. Santaella et al., New J. Chem., 1992, Vol. 16, pp. 399-404;

A. Milius et al., New J. Chem., 1992, Vol. 16, pp. 771-773;

F. Szönyi et-al., Journal of Fluorine Chemistry, 1991, Vol. 55, pp. 85-92;

C. Santaella et al., Angew. Chem., 1991, Vol. 103, No. 5, pp. 584-586;

M.-P. Krafft et al., Angew. Chem., 1993 (Vol. 105, No. 5, pp. 783-785; EP 0 548 096 B1.

The new diamagnetic perfluoroalkyl-containing substances are also the subject of this invention. Their production is carried out analogously to the above-mentioned compounds that are known in the literature and is described in the examples below.

In this case, these are substances of general formula XVII
R^{F}—X¹ (XVII)
in which RF represents a straight-chain or branched perfluoroalkyl radical with 4 to 30 carbon atoms, and X¹ is a radical that is selected from the group of the following radicals (in this case, n is a number between 1 and 10):

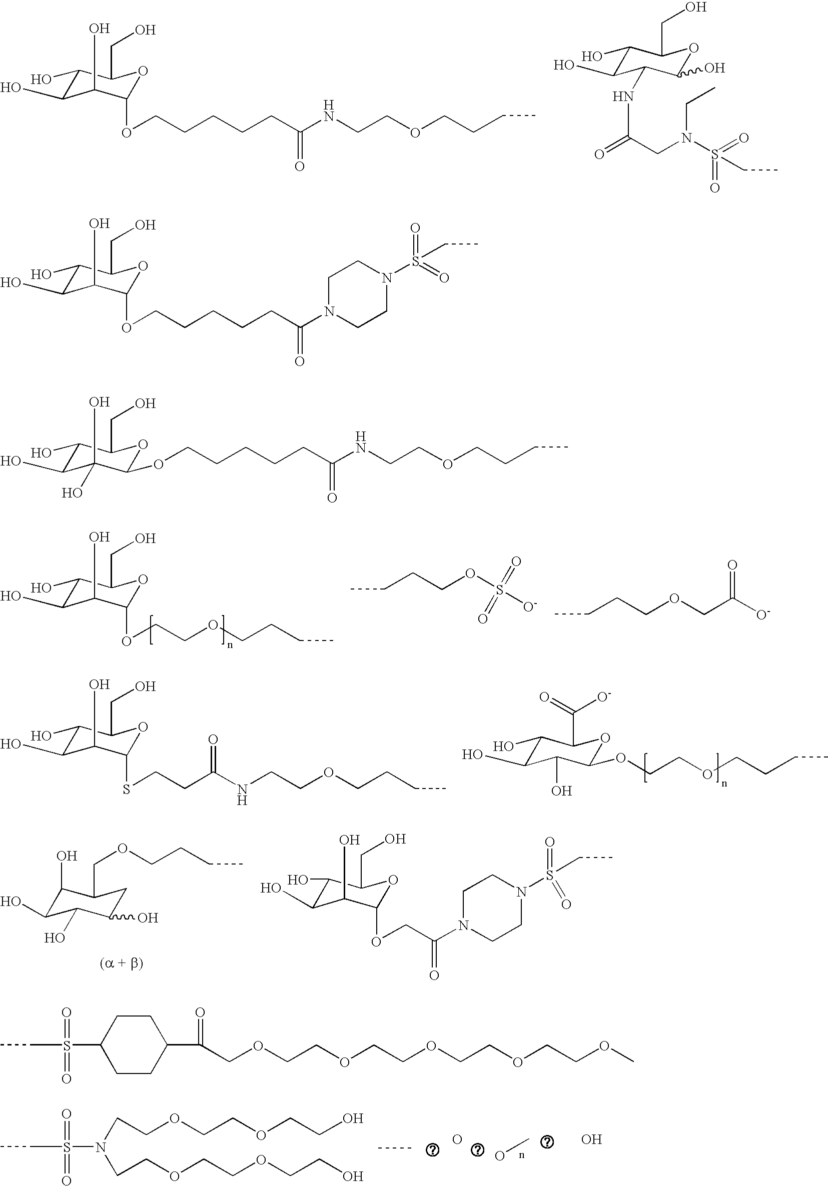

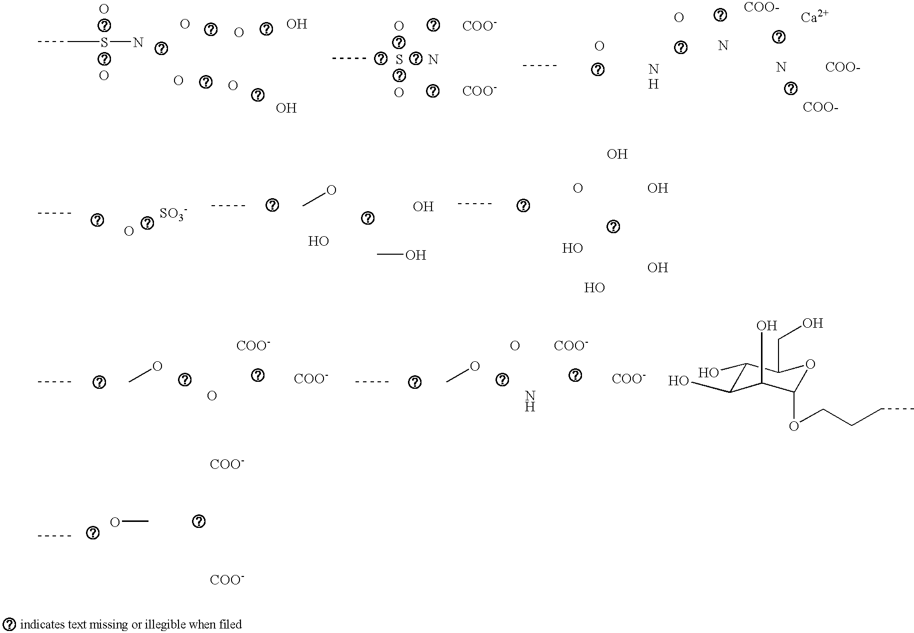

Preferred diamagnetic perfluoroalkyl-containing substances are those with a mnonosaccharide as hydrophilic group B².

Especially preferred diamagnetic perfluoroalkyl-containing compounds contain a perfluoroalkyl radical R_{f} with 6 to 12 carbon atoms, a linker L², which represents an —SO₂ group or a straight-chain or branched carbon chain with up to 20 carbon atoms, which in turn contains one or more —O—, —CO—, —CONH—, —NHCO—, —CONR—, NRCO—, —SO₂ groups or a piperazine, in which R has the above-indicated meaning, and a monosaccharide as hydrophilic group B².

Other suitable diamagnetic perfluoroalkyl-containing compounds are conjugates that consist of cyclodextrin and perfluoroalkyl-containing compounds. These conjugates consist of α-, β-, or γ-cyclodextrin and compounds of general formula XVIII
A¹-L³-R^{F} (XVIII)

in which A¹ stands for an adamantane, biphenyl or anthracene molecule, L³ stands for a linker and RF stands for a straight-chain or branched perfluoroalkyl radical with 4 to 30 carbon atoms. Linker L3 is a straight-chain hydrocarbon chain with 1 to 20 carbon atoms, which can be interrupted by one or more oxygen atoms, one or more CO—, SO₂—, CONH—, NHCO—, CONR—, NRCO—, NH—, NR groups or a piperazine, whereby R is a C₁-C₅ alkyl radical.

Preferred compounds are the following compounds:

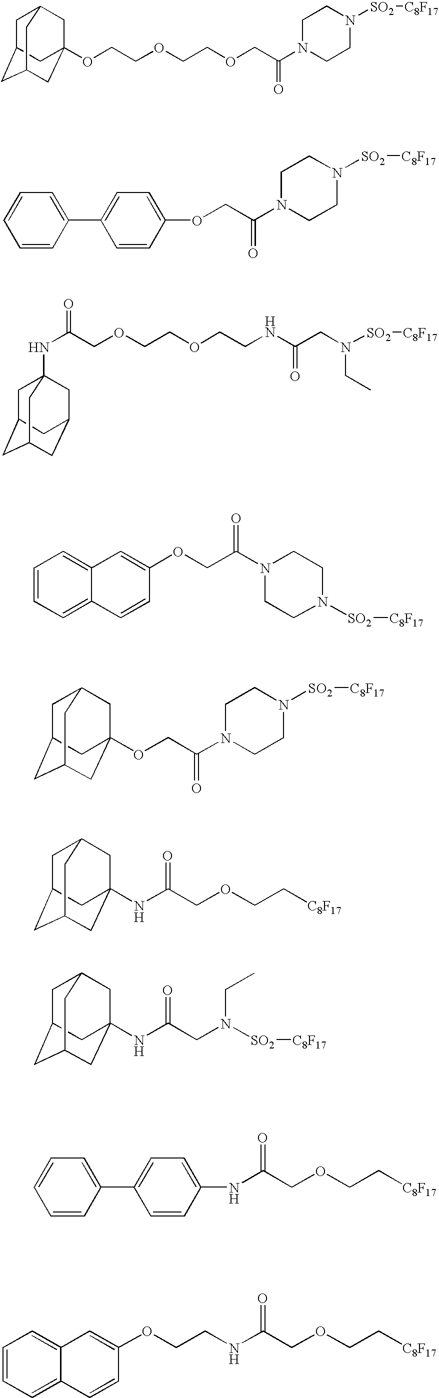

The galenical formulations of this invention contain the paramagnetic and diamagnetic perfluoroalkyl-containing compounds in a mixing ratio of between 5:95 and 95:5. Preferred are mixing ratios of between 40:60 and 60:40 of the two substances. Both substances are used in millimolar concentrations. Concentrations of between 0.5 and 1000 mmol/l of solvent are reached. The solvent is preferably water. The metal concentration of the formulations is preferably in a range of 50-250 mmol/l, since only then are diagnostically relevant images obtained in nuclear spin tomography.

Preferred are mixtures of paramagnetic and diamagnetic perfluoroalkyl-containing compounds, in which the perfluoroalkyl chains have a/length of 6 to 12 carbon atoms. Especially preferred are mixtures in which both the paramagnetic and the diamagnetic perfluoroalkyl-containing compounds have a perfluoroalkyl chain with 8 carbon atoms.

The new galenical formulations show surprising advantages in their use as contrast media in nuclear spin tomography. Compared to the already known contrast media, they show an improved compatibility and an almost complete excretion. The local compatibility further is also higher than in the previously known contrast media, and the new formulations simultaneously show a higher organ specificity. The concentration in the lymph nodes is higher than in the known contrast media for lymphography. This results in a higher relaxivity and thus in an improved imaging.

The production of the galenical formulations is carried out in that the paramagnetic perfluoroalkyl-containing compounds (component A) and the diamagnetic perfluoroalkyl-containing substances (component B) are weighed in molar fractions between 0.05 and 0.95 in component A or B and are dissolved in a suitable solvent. An especially suitable solvent is water. Commonly used galenical additives, such as, e.g., buffer solutions and the Ca-salt of the complexing agent, are then added in excess to this solution. At 10 to 100° C.; the solutions are stirred vigorously. As an alternative, the solutions can be treated at 10 to 100° C in an ultrasound bath. Another alternative consists in that the solutions are treated with microwaves.

In substances that do not dissolve in water as individual components, it has proven advantageous to add a solubilizer, such as alcohol (e.g., methanol or ethanol) or another water-miscible solvent and then to distill off the latter slowly. The distillation can take place under vacuum. The residue is then dissolved in water, and the solution is filtered. It is also possible to dissolve each component per se separately in a solvent, then to join them and to proceed further as above. It has proven advantageous to introduce a relatively strongly concentrated solution (>100 mmol) of the metal complex (component A) and then to add component B in a pure state and to treat the solution to stirring or with ultrasound or microwaves as mentioned above.

In humans, the galenical formulations of this invention can be injected locally (either subcutaneously or directly percutaneously into the tissue of interest). Several injection sites (weals) with-a respective injection volume of 0.2 to 1 ml grouped around the area in question (e.g., tumor) are possible. In this case, the total injected volume should not in any case exceed 5 ml. This means that in the formulation, a metal concentration of 50-250 mmol/l must be present, so that a potential clinical dose of 5-10 μmol/kg of body weight can be administered with this volume. The site of administration depends on whether staining is to be done specifically to a certain lymph drainfield from the tissue that corresponds to it (e.g., in the case of gynecological or rectal tumors) or whether the unknown drainfield of a certain lesion (ergo the area for a possible therapeutic intervention, e.g., in melanoma or breast cancer) is to be visualized. To obtain clinically relevant information on the lymph node status, e.g., in the case of malignant tumors, a concentration on three successive lymph node stations with relatively uniform dispersion (generally a drop in concentration between the first and third station that is not larger than a factor of 3-4) is desirable. For the MRT imaging, gadolinium concentrations of at least 50 μmol/l and at most 2500 μmol/l are required in normal lymph node tissue, where the concentration of the compound is carried out. The imaging can (depending on injection site and tissue) be carried out after 30 minutes and is then possible for another 4 to 6 hours after injection. Since mainly the T1-relaxation times of the lymph node tissue are influenced with the compounds of the gadolinium complexes according to the invention, fast T1-weighted sequences (e.g., gradient echo sequences with TR of 10-20 ms, TE of 5 ms and flip angles of 40-80°) are best able to detect an MRT-enhancement of the lymph node stations. Since lymph nodes are very frequently embedded in fatty tissue, and the latter has a very high signal intensity in such sequences, fat-suppressed measuring methods are also worth considering. Compared to formulations of superparamagnetic iron oxide particles, paramagnetic gadolinium complexes in connection with T1-weighted measuring sequences have the great advantage that they allow MRT images with higher spatial resolution, with smaller distorsion artifacts (based on susceptibility artifacts) and with shorter recording time. Since a positive labeling of the lymph nodes is carried out (i.e., a signal increase), MRT pictures without contrast media-are no longer absolutely necessary for comparison, and the overall examination time per patient can be shortened.

The galenical formulations according to the invention are especially suitable as contrast media for nuclear spin tomography. In addition to the visualization of the lymph nodes, the visualization of the blood-pool can also be carried out with the galenical formulations according to the invention.

The examples below explain the subject of the invention, without intending that it is to be limited to these examples.

The Production of Metal Complexes I-V That are Used, Described in

| Complex | Bibliographic Reference |
|---|---|
| I | WO 97/26017 |
| | Gadolinium complex of 10-[1- |
| | methyl-2-oxo-3-aza-5-oxo-5-{4- |
| | perfluoro-octylsulfonyl- |
| | piperazin-1-yl}-pentyl]-1,4,7- |
| | tris(carboxymethyl)-1,4,7,10- |
| | tetraazacyclododecane |
| II | WO 97/26017 |
| | Gadolinium complex of 10-[2- |
| | hydroxy-4-aza-5-oxo-7-oxa- |
| | 10,10,11,11,12,12,13,13,14,14, |
| | 15,15,16,16,17,17,17- |
| | heptadecafluoroheptadecyl]- |
| | 1,4,7-tris(carboxy-methyl)- |
| | 1,4,7,10-tetraazacyclododecane |
| III | DE 197 29 013 |
| | 1,4,7-Tris{1,4,7-tris(N- |
| | carboxylatomethyl)-10-(N-1- |
| | methyl-3,6-diaza-2,5,8-trioxo- |
| | octane-1,8-diyl)1,4,7,10- |
| | tetraazacyclododecane, Gd- |
| | complex}-10-(N- |
| | 2H,2H,4H,4H,5H,5H-3-oxa- |
| | perfluoro-tridecanoyl)- |
| | 1,4,7,10-tetraazacyclododecane |
| IV | DE 197 29 013 |
| | 1,4,7-Tris{1,4,7-tris(N- |
| | carboxylatomethyl)-10-(N-1- |
| | methyl-3-aza-2,5-dioxo- |
| | pentane-1,5-diyl]-1,4,7,10- |
| | tetraazacyclododecane, Gd- |
| | complex}-10-[2-(N-ethyl-N- |
| | perfluorooctylsulfonyl)- |
| | amino]-acetyl-1,4,7,10- |
| | tetraazacyclododecane |
| V | WO 97/26017 |
| | Gadolinium complex of 10-[2- |
| | hydroxy-4-aza-5-oxo-7-aza-7- |
| | (perfluorooctyl-sulfonyl)- |
| | nonyl]-1,4,7- |
| | tris(carboxymethyl)-1,4,7,10- |
| | tetraazacyclododecane |

Synthesis of Metal Complexes VI-IX
Metal Complex VI

### a) 2H,2H,4H,4H,5H,5H-3-Oxa)-perfluorotridecanoic acid-N-(2,3-dihydroxypropyl)-amide

8.90 g (70 mmol) of oxalyl chloride is added to 30 g (57.45 mmol) of 2H,2H,4H,4H,5H,5H-3-oxa-perfluorotridecanoic acid in 300 ml of dichloromethane, and it is stirred for 12 hours at room temperature. It is evaporated to the dry state in a vacuum. The residue is dissolved in 100 ml of dichloromethane and added in drops at 0° C. to a solution of 5.47 g (60 mmol) of 2,3-dihydroxypropylamine and 6.07 g (60 mmol) of triethylamine, dissolved in 200 ml of dichloromethane. It is stirred for 3 hours at 0° C., then for 6 hours at room temperature. 300 ml of 5% aqueous hydrochloric acid is added, and it is thoroughly stirred for 15 minutes. The organic phase is separated, dried on magnesium sulfate and evaporated to the dry state in a vacuum. The residue is chromatographed on silica gel (mobile solvent: dichloromethane/ethanol=15:1).

Yield: 29.70 g (87% of theory) of a colorless solid

Elementary analysis:

Cld: C, 30.32; H, 2.20; N, 2.36; F, 54.35.

Fnd: C, 30.12; H, 2.41; N, 2.18; F, 54.15.

### b) N-(2,3-Dihydroxypropyl)-N-1H,1H,2H,2H,4H,4H,5H,5H-3-oxa-perfluorotridecyl)-amine

30 g (48.8 mmol) of the title compound of Example VIa is dissolved in 300 ml of tetrahydrofuran, and 50 ml of 10 M borane dimethyl sulfide (in tetrahydrofuran) is added. It is refluxed for 16 hours. It is cooled to 0° C., and 300 ml of methanol is added in drops, then it is evaporated to the dry state in a vacuum. The residue is taken up in a mixture of 300 ml of ethanol/50 ml of 10% aqueous hydrochloric acid and stirred for 8 hours at 60° C. It is evaporated to the dry state in a vacuum, the residue is taken up in 300 ml of 5% aqueous sodium hydroxide solution and extracted three times with 300 ml of dichloromethane each. The organic phases are dried on magnesium sulfate, evaporated to the dry state in a vacuum, and the residue is chromatographed on silica gel (mobile solvent: dichloromethane/methanol=15:1).

Yield: 24.07 g (85% of theory) of a colorless solid

Elementary analysis:

Cld: C, 31.05; H, 2.61; N, 2.41; F, 55.66.

Fnd: C, 31.91; H, 2.78; N, 2.33; F, 55.47.

### c) 1,4,7-Tris(carboxylatomethyl)-10-[(3-aza-4-oxo-hexan-5-ylic)-acid-N-(2,3-dihydroxypropyl)-N-(1H,1H,2H,2H,4H,4H, 5H,5H-3-oxa)-perfluorotridecyl)-amide]-1,4,7,10-tetraazacyclododecane, gadolinium complex

10 g (15.88 mmol) of the gadolinium complex of 10-[1-(carboxymethylcarboamoyl)-ethyl]-1,4,7,10-tetraazacyclododecane-1,4,7-triacetic acid and 1.35 g (31.76 mmol) of lithium chloride are dissolved at 60° C. in 100 ml of dimethyl sulfoxide. It is cooled to 15° C., and 9.21 (15.88 mmol) of the title compound of Example VIb is added. It is stirred for 10 minutes, and then 7.42 g (30,mmol) of 2-ethoxy-1-ethoxycarbonyl-1,2-dihydroquinoline is added. It is stirred for 12 hours at room temperature. The solution is poured into a mixture of 200 ml of acetone/1300 ml of diethyl ether and stirred for 2 hours at room temperature. The deposited precipitate is filtered off, dissolved in a mixture that consists of a little ethanol/water and chromatographed on silica gel RP-18 (mobile solvent: gradient that consists of tetrahydrofuran/acetonitrile/water).

Yield: 16.09 g (85% of theory) of a colorless, amorphous powder

Water content: 6.3%

Elementary analysis (relative to anhydrous substance):

Cld: C, 34.26; H, 3.64; N, 7.05; F, 27.10; Gd, 13.19.

Fnd: C, 34.12; H, 3.83; N, 6.91; F, 26.88; Gd, 12.93.

Metal Complex VII

### 1,4,7-Tris(carboxylatomethyl)-10-[(3-aza-4-oxo-hexan-5-ylic)-acid-N-(1H,1H,2H,2H,4H,4H,5H,5H-3-oxa-perfluorotridecyl)-amide]-1,4,7,10-tetraazacyclododecane, gadolinium complex

10 g (15.88 mmol) of the gadolinium complex of 10-[1-(carboxymethylcarboamoyl)-ethyl]-1,4,7,10-tetraazacyclododecane-1,4,7-triacetic acid and 1.35 g (31.76 mmol) of lithium chloride and 3.66 g (31.76 mmol) of N-hydroxysuccinimide are dissolved at 60° C. in 100 ml of dimethyl sulfoxide. It is cooled to 15° C., and 3.51 (17 mmol) of N,N′-dicyclohexylcarbodiimide is added and stirred for 5 hours at 15° C. To separate the urea, the solution is filtered. 8.63 g (15.88 mmol) of the title compound of Example VIIIb and 5.06 g (50 mmol) of triethylamine are added to the filtrate and stirred for 12 hours at room temperature. The solution is poured into 1,500 ml of diethyl ether/100 ml of acetone and stirred for 30 minutes. The precipitated solid is filtered off and chromatographed on silica gel RP-18. (mobile solvent: gradient that consists of tetrahydrofuran/acetonitrile/water).

Yield: 13.86 g (78% of theory) of a colorless, amorphous powder

Water content: 9.3%

Elementary analysis (relative to anhydrous substance):

Cld: C, 33.28; H, 3.42; N, 7.51; F, 28.87; Gd, 14.05.

Fnd: C, 33.12; H, 3.61; N, 7.37; F, 28.69; Gd, 13.89.

Metal Complex VIII

### a) 2H,2H,4H,4H,5H,5H-3-Oxa-perfluorotridecanoic acid amide

8.90 g (70 mmol) of oxalyl chloride is added to 30 g (57.45 mmol) of 2H,2H,4H,4H,5H,5H-3-oxa-perfluorotridecanoic acid in 300 ml of dichloromethane and stirred for 12 hours at room temperature. It is evaporated to the dry state in a vacuum. The residue is dissolved in 200 ml of dichloromethane. Then, ammonia gas is introduced into the solution for about 2 hours at 0° C. It is stirred for 4 more hours at 0° C., and then for 2 hours at room temperature. 300 ml of 5% aqueous hydrochloric acid is added, and it is stirred thoroughly for 15 minutes. The organic phase is separated, dried on magnesium sulfate and evaporated to the dry state in a vacuum. The residue-is chromatographed on silica gel (mobile solvent: dichloromethane/acetone=20:1).

Yield: 27.85 g (93% of theory)

Elementary analysis:

Cld: C, 27.66; H, 1.55; N, 2.69; F, 61.97.

Fnd: C, 27.49; H, 1.72; N, 2.54; F, 61.81.

### b) 1H,1H,2H,2H,4H,4H,5H,5H-3-Oxa-perfluorotridecylamine, hydrochloride

27 g (51.8 mmol) of the title compound of Example VIIIa is dissolved in 300 ml of tetrahydrofuran, and 31 ml of 10 m borane dimethyl sulfide (in tetrahydrofuran) is added. It is refluxed for 16 hours. It is cooled to 0° C., and 200 ml of methanol is added in drops, then evaporated to the dry state in a vacuum. The residue is taken up in a mixture of 400 ml of ethanol/100 ml of 10% aqueous hydrochloric acid, and it is stirred for 8 hours at 60° C. It is evaporated to the dry state in a vacuum, and the residue is recrystallized from a little ethanol/diethyl ether.

Yield: 26.75 g (95% of theory) pf a colorless, crystalline solid

Elementary analysis:

Cld: C, 26.51; H, 2.04; N, 2.58; F, 59.41; Cl, 6.52.

Fnd: C, 26.37; H, 2.21; N, 2.46; F, 59.25; Cl, 6.38.

### c) 3,6,9,12,15-Pentaoxyhexadecanoic acid-N-(1H,1H,2H,2H,4H,4H,5H,5H-3-oxa)-perfluorotridecyl)-amide

14.24 g (50 mmol) of 3,6,9,12,15-pentaoxahexadecanoic acid chloride is added in drops at 0° C. to 26.5 g (48.74 mmol) of the title compound of Example VIIIb and 14.8 g (146.2 mmol) of triethylamine, dissolved in 300 ml of dichloromethane, and it is stirred for 3 hours at 0° C. 300 ml of 5% aqueous hydrochloric acid is added, and it is stirred thoroughly for 30 minutes. The organic phase is separated, dried on magnesium sulfate and evaporated to the dry state in a vacuum. The residue is chromatographed on silica gel (mobile solvent: dichloromethane/acetone: 20:1).

Yield: 32.03 g (87% of theory) of a colorless oil

Elementary analysis:

Cld: C, 36.57; H, 4.00; N, 1.85; F, 42.75.

Fnd: C, 36.46; H, 4.12; N, 1.76; F, 42.53.

### d) N-(3,6,9,12,15-Pentaoxahexadecyl)-N-(1H,1H,2H,2H,4H,4H-3-oxa)-perfluorotridecyl)-amide

31 g (41.03 mmol) of the title compound of Example VIIIc is dissolved in 300 ml of tetrahydrofuran, and 25 ml of 10 M boranedimethyl sulfide (in tetrahydrofuran) is added. It is refluxed for 16 hours. It is cooled to 0° C., and 200 ml of methanol is added in drops, then it is evaporated to the dry state iv. The residue is taken up in a mixture of 300 ml of ethanol/50 ml of 10% aqueous hydrochloric acid, and it is stirred for 8 hours at 40° C. It is evaporated to the dry state in a vacuum, the residue is taken up in 300 ml of 5% aqueous sodium hydroxide solution and extracted 3 times with 300 ml of dichloromethane each. The organic phases are dried on magnesium sulfate, evaporated to the dry state in a vacuum, and the residue is chromatographed on silica gel (mobile -solvent: dichloromethane/2-propanol=15:1).

Yield: 27.68 g-(91% of theory)

Elementary analysis:

Cld: C, 37.26; H, 4.35; N, 1.89; F, 43.56.

Fnd: C, 37.11; H, 4.51; N, 1.73; F, 43.41.

### e) 1,4,7-Tris(carboxylatomethyl)-10-{(3-aza-4-oxo-hexan-5-ylic)-acid-[N-3,6,9,12,15-pentaoxa)-hexadexyl)-N-(1H,1H,2H,2H,4H,4H,5H,5H-3-oxa)-perfluorotridecyl]-amide}-1,4,7,10-tetraazacyclododecane, gadolinium complex

10 g (15.88 mmol) of the gadolinium complex of 10-[1-(carboxymethylcarboamoyl)-ethyl]-1,4,7,10-tetraazacyclododecane-1,4,7-triacetic acid and 1.35 g (31.76 mmol) of lithium chloride are dissolved at 60° C. in 100 ml of dimethyl sulfoxide. It is cooled to 15° C., and 11.77 (15.88 mmol) of the title compound of Example VIIId is added. It is stirred for 10 minutes, and then 7.42 g (30 mmol) of 2-ethoxy-1-ethoxycarbonyl-1,2-dihydroquinoline is added. It is stirred for 12 hours at room temperature. The solution is poured into a mixture of 200 ml of acetone/1300 ml of diethyl ether and stirred for 2 hours at room temperature. The deposited precipitate is filtered off, dissolved in a mixture that consists of a little ethanol/water and chromatographed on silica gel RP-18 (mobile solvent: gradient that consists of tetrahydrofuran/acetonitrile/water).

Yield: 18.05 g (84% of theory) of a colorless, amorphous powder

Water content: 6.2%

Elementary analysis (relative to anhydrous substance):

Cld: C, 37.28; H, 4.47; N, 6.21; F, 23.87; Gd, 11.62.

Fnd: C, 37.11; H, 4.61; N, 6.03; F, 23.64; Gd, 11.42.

Metal Complex IX

### a) 2H,2H,4H,4H,5H,5H-3-Oxa-perfluorotridecanoic acid-N-(5-hydroxy-3-oxa-pentyl)-amide

8.90 g (70 mmol) of oxalyl chloride is added to 30 g (57.45 mmol) of 2H,2H,4H,4H,5H,5H-3-oxa-perfluorotridecanoic acid in 300 ml of dichloromethane, and it is stirred for 12 hours at room temperature. It is evaporated to the dry state in a vacuum. The residue is dissolved in 100 ml of dichloromethane and added in drops at 0° C. to a solution of 6.25 g (60 mmol) of 5-hydroxy-3-oxa-pentylamine and 6.07 g (60 mmol) of triethylamine, dissolved in 200 ml of dichloromethane. It is stirred for 3 hours at 0° C., then for 6 hours at room temperature. 300 ml of 5% aqueous hydrochloric acid is added and stirred thoroughly for 15 minutes. The organic phase is separated, dried on magnesium sulfate and evaporated to the dry state in a vacuum. The residue is chromatographed on silica gel (mobile solvent: dichloromethane/acetone=15:1).

Yield: 32.20 g (92% of theory) of a colorless solid

Elementary analysis:

Cld: C, 31.54; H, 2.65; N, 2.30; F, 53.01.

Fnd: C, 31.61; H, 2.84; N, 2.14; F, 52.85.

### b) N-(5-Hydroxy-3-oxa-pentyl)-N-(1H,1H,2H,2H,4H,4H,5H,5H-3-oxa-perfluoro-tridecyl)-amine

30 g (49.24 mmol) of the title compound of Example IXa is dissolved in 300 ml of tetrahydrofuran, and 31 ml of 10 M borane dimethyl sulfide (in tetrahydrofuran) is added. It is refluxed for 16 hours. It is cooled to 0° C., and 200 ml of methanol is added in drops, then it is evaporated to the dry state in a vacuum. The residue is taken up in a mixture of 300 ml of ethanol/50 ml of 10% aqueous hydrochloric acid and stirred for 10 hours at 50° C. It is evaporated to the dry state in a vacuum, the residue is taken up in 300 ml of 5% aqueous sodium hydroxide solution and extracted three times with 300 ml of dichloromethane each. The organic phases are dried on magnesium sulfate, evaporated to the dry state in a vacuum, and the residue is chromatographed on silica gel (mobile solvent: dichloromethane/2-propanol=20:1).

Yield: 26.09 g (89% of theory) of a colorless solid

Elementary analysis:

Cld: C, 32.28; H, 3.05; N, 2.35; F, 54.25.

Fnd: C, 32.12; H, 3.21; N, 2.18; F, 54.09.

### c) 1,4,7-Tris(carboxylatomethyl)-10-[(3-aza-4-oxo-hexan-5-ylic)-acid-N-(5-hydroxy-3-oxa-pentyl)-N-(1H,1H,2H,2H,4H,4H,5H,5H-3-oxa)-perfluorotridecyl)-amide]-1,4,7,10-tetraazacyclododecane, gadolinium complex

10 g (15.88 mmol) of the gadolinium complex of 10-[1-(carboxymethylcarboamoyl)-ethyl]-1,4,7,10-tetraazacyclododecane-1,4,7-triacetic acid and 1.35 g (31.76 mmol) of lithium chloride are dissolved at 60° C. in 100 ml of dimethyl sulfoxide. It is cooled to 15° C., and 9.45 (15.88 mmol) of the title compound of Example IXb is added. It is stirred for 10 minutes, and then 7.42 g (30 mmol) of 2-ethoxy-1-ethoxycarbonyl-1,2-dihydroquinoline is added. It is stirred for 12 hours at room temperature. The solution is poured into a mixture of 200 ml of acetone/1300 ml of diethyl ether and stirred for 2 hours at room temperature. The deposited precipitate is filtered off, dissolved in a mixture that consists of a little ethanol/water and chromatographed on silica gel RP-18 (mobile solvent: gradient that consists of tetrahydrofuran/acetonitrile/water).

Yield: 16.10 g (84% of theory) of a colorless, amorphous powder

Water content: 5.7%

Elementary analysis (relative to anhydrous substance):

Cld: C, 34.83; H, 3.84; N, 6.96; F, 26.76; Gd, 13.03.

Fnd: C, 34.65; H, 3.96; N, 6.84; F, 26.62; Gd, 12.91.

### EXAMPLE 1

### a) 1,2,3,4,6-Penta-O-acetyl-α,β-D-mannopyranose

Analogously, as described in the literature [M. L. Wolfrom and A. Thompson in Methods in Carbohydrate Chemistry (R. L. Whistler, M. L. Wolfrom and J. N. BeMiller, Eds.), Academic Press, New York, Vol. II, 53, pp. 211-215, (1963)], the reaction of 150 g (832.5 mmol) of α,β-D-mannopyranose with a mixture that consists of 1,500 ml of absolute pyridine and 1,500 ml of acetic acid anhydride provides, after working-up, 315 g (96.7%) of the above-mentioned title compound as a crude product in the form of a viscous and colorless oil. By ¹H-NMR-spectroscopic examination of the title compound that is thus obtained, the α to β-ratio of both anomers was found to be 4:1. A separation of the α,β-anomers of the above-mentioned title compound can be eliminated for performing the reaction steps below.

Elementary analysis:

Cld: C, 49.21; H, 5.68.

Fnd: C, 49.12; H, 5.78.

### b) 6-[1-O-α-(2,3,4,6-Tetra-O-acetyl-D-mannopyranosyl)-hexanoic acid ethyl easter]

Analogously, as described in the literature for the synthesis of aryl glycopyranosides [J. Conchie and G. A. Levvy in Methods in Carbohydrate Chemistry (R. L. Whistler, M. L. Wolfrom and J. N. BeMiller, Eds.), Academic Press, New York, Vol. II, 90, pp. 345-347, (1963)], the reaction of 156.2 g (400 mmol) of the title compound of Example Ia) as an α,β-anomer mixture with 67 ml (400 mmol) of 6-hydroxy-hexanoic acid ethyl ester and 60.8 ml (520 mmol) of tin(IV) chloride in a total of 600 ml of 1,2-dichloroethane results in the formation of 100.05 g (51% of theory) of the above-mentioned title compound as a colorless and viscous oil after column-chromatographic purification (eluant: hexane/ethyl acetate 2:1). ¹H-NMR-spectroscopic examination of the title compound that is thus obtained showed that the above-mentioned title compound is only the pure α-anomer.

Elementary analysis:

Cld: C, 52.94; H, 6.77.

Fnd: C, 52.80; H, 6.78.

### c) 6-[1-O-α-(2,3,4,6-Tetra-O-benzyl-D-mannopyranosyl)-hexanoic acid

A stirred suspension of 141.0 g (289 mmol) of the title compound of Example Ib) in 200 ml of dioxane is mixed at room temperature and with simultaneous vigorous stirring in portions with a total of 238.5 g (4.26 mol) of finely powdered potassium hydroxide powder. To increase the stirrability, the reaction mixture is mixed with another 200 ml of dioxane, and the suspension that is thus obtained is subsequently heated to boiling and mixed drop by drop at this temperature with a total of 372 ml (3.128 mol) of benzyl bromide over a period of two hours. After a reaction time of 4 hours at 110° C. followed by 12 hours at room temperature, the reaction mixture is slowly poured into a total of 2.5 liters of ice water for the purpose of working-up, and the water phase is subsequently completely extracted with diethyl ether. After the ether phase that is thus obtained is washed and the same is subsequently dried on sodium sulfate, salt is suctioned out, and the diethyl ether is drawn off in a vacuum. Excess benzyl bromide is then quantitatively distilled off from the reaction mixture in an oil pump vacuum at an oil bath temperature of 180° C. The resinous-oily residue that is thus obtained is purified on silica gel with use of ethyl acetate/hexane (1:10) as an eluant.

Yield: 172.2 g (91.0% of theory) of the above-mentioned title compound in the form of a colorless and extremely viscous oil

Elementary analysis:

Cld: C, 75.68; H, 7.16.

Fnd: C, 75.79; H, 7.04.

### d) 6-(1-O-α-(2,3,4,6-Tetra-O-benzyl-D-mannopyranosyl)-hexanoic acid-N-(3-oxa-1H,1H,2H,2H,4H,4H,5H,5H-perfluorotridecyl)-amide

100 g (134 mmol) of the acid that is described in Example Ic) and 13.5 g (134 mmol) of triethylamine are dissolved in 1,200 ml of dry tetrahydrofuran. After cooling to −15° C., a solution of 18.45 g (135 mmol) of isobutyl chloroformate in 200 ml of dry tetrahydrofuran is slowly added in drops while being stirred, whereby the internal temperature does not exceed −10° C. After a reaction time of 15 minutes at −15° C., a solution of 165.5 g (134 mmol) of 1-amino-1H,1H,2H,2H-perfluorodecane and 13.5 g (134 mmol) of triethylamine in 250 ml of dry tetrahydrofuran is added in drops at −20° C. After a reaction time of one hour at −15° C. and two hours at room temperature, the reaction solution is evaporated to the dry state in a vacuum. The remaining residue is taken up in 300 ml of ethyl acetate and washed twice with 400 ml of saturated sodium bicarbonate solution each-and once with 500 ml of water. After the organic phase is dried on sodium sulfate, salt is suctioned out, and the ethyl acetate is drawn off in a vacuum. The remaining oily residue is purified on silica gel with use of dichloromethane/hexane/2-propanol (10:5:1) as an eluant.

Yield: 143.8 g (86.9% of theory)

Elementary analysis:

Cld: C, 57.38; H, 4.98; N, 1.13; F, 26.15.

Fnd: C, 57.30; H, 5.44; N, 1.01; F, 26.25.

### e) 6-[1-O-α-D-Mannopyranosyl)-hexanoic acid N-(3-oxa-1H,1H,2H,2H,4H,4H,5H,5H-perfluorotridecyl)-amide

40.0 g (32.38 mmol) of the title compound of Example 1d) is dissolved in 750 ml of 2-propanol and mixed with 2.0 g of palladium catalyst (10% Pd/C). The reaction solution is hydrogenated for 12 hours at 22° C. and 1 atmosphere of hydrogen pressure. Then, catalyst is filtered out, and the filtrate is evaporated to the dry state. The remaining residue is taken up in 300 ml of dimethyl sulfoxide, and 21.52 g (88.0% of theory) of the above-mentioned title compound is obtained as a colorless and crystalline powder with the decomposition melting point of 88.5° C. from the product solution that is thus obtained by mixing with a total of 1000 ml of diethyl ether after the precipitated solid is suctioned off.

Elementary analysis:

Cld: C, 36.01; H, 5.92; N, 1.75; F, 40.34.

Fnd: C, 36.07; H, 6.08; N, 1.76; F, 40.66.

### f) Production of a formulation of gadolinium complex I and 6-[1-O-α-D-mannopyranosyl)-hexanoic acid N-(3-oxa-1H,1H,2H,2H,4H,4H,5H,5H-perfluorotridecyl)-amide

3.17 g (4.2 mmol) of the title compound of Example 1e is added to 35 ml of a solution of gadolinium complex I (280 mmol/L) that is dissolved in 0.45% aqueous common salt solution (pH 7.4:0.25 mg/L of CaNa₃DTPA), and, with a 0.9% aqueous common salt solution, it yields a total of 98 ml. It is heated for 2 hours at 60° C. in an ultrasound bath. The solution is cooled to room temperature and set at pH 7.4 with aqueous 2N sodium hydroxide solution. It is filtered through a 0.2 μm filter, and the filtrate is decanted into-vials. A solution that is thus produced can be used directly for biological experiments. (The concentration is 100 mmol of Gd/L).

### EXAMPLE 2

### a) 1-O-α-D-[(1-Perfluorooctylsulfonylpiperazine-4-carbonyl)-pentyl-5]-2,3,4,6-tetra-O-benzyl-mannopyranose

74.59 g (100 mmol) of the acid that is described in Example 1c) and 10.11 g (100 mmol) of triethylamine are dissolved in 800 ml of a mixture of tetrahydrofuran/acetonitrile (mixing ratio 7:3). Then, it is mixed drop by drop at room temperature with 500 ml of a tetrahydrofuran solution of 58.0 g (102.0 mmol) of 1-perfluorooctyl-sulfonylpiperazine; 10.11 g (100 mmol) of triethylamine and 16.84 g (110 mmol) of 1-hydroxybenzotriazole. The reaction solution that is thus obtained is mixed at −5° C. with a solution of 22.7 g (110 mmol) of dicyclohexylcarbodiimide, dissolved in 100 ml of tetrahydrofuran, and then stirred at −5° C. for two more hours. After the reaction solution has thawed, it is stirred at room temperature for another 12 hours, precipitated dicyclohexylurea is filtered out, and the filtrate that is obtained is evaporated to the dry state in a vacuum. The remaining residue is taken up in 600 ml of ethyl acetate and washed twice with 300 ml of saturated sodium bicarbonate solution each as well as twice with 300 ml of water each. After the organic phase is dried on sodium sulfate, salt is suctioned out, and the ethyl acetate is drawn off in a vacuum. The remaining oily residue is purified on silica gel with use of dichloromethane/acetone/2-propanol (16:2:1) as an eluant.

Yield: 113.01 g (79.8% of theory) of a colorless and viscous oil

Elementary analysis:

Cld: C, 58.52; H, 4.27; N, 1.98; S, 2.26; F, 22.80.

Fnd: C, 58.42; H, 4.41; N, 1.80; S, 2.28; F, 23.02.

### b) 1-O-α-D-[(1-Perfluorooctylsulfonyl-piperazine-4-carbonyl)-pentyl-5]-mannopyranose

50 g (35.30 mmol) of the title compound of Example 2a) is dissolved in a mixture that consists of 500 ml of 2-propanol and 50 ml of water, and 2 g of palladium catalyst (10% Pd on activated carbon) is added. It is hydrogenated for 12 hours at room temperature. Catalyst is filtered out, and the filtrate is evaporated to the dry state in a vacuum. The residue is dissolved in 200 ml of methanol, and the reaction product is precipitated by mixing with a total of 800 ml of diethyl ether. After the solid that is thus obtained is suctioned off, the latter is dried in a vacuum at 50° C.

Yield: 29.51 g (99% of theory) of an amorphous solid

Elementary analysis:

Cld: C, 34.13; H, 3.46; N, 3.32; S, 3.80; F, 38.23.

Fnd: C, 34.28; H, 3.81; N, 3.25; S, 3.80; F, 38.01.

### c) Production of a formulation of gadolinium complex II and 1-O-α-D-[(1-perfluorooctylsulfonyl-piperazine-4-carbonyl)-pentyl]-5]-mannopyranose

9.92 g (11.75 mmol) of the title compound of Example 2b is added to 47 ml of a solution of gadolinium complex II (250 mmol/L) that is dissolved in 0.45% of aqueous sodium chloride solution), and it is heated for 10 minutes in the microwave. The solution is cooled to room temperature, filtered though a 0.2 μm filter, and the filtrate is decanted into vials. A solution that is thus produced can be used directly for biological experiments. (The concentration is 250 mmol of Gd/L.)

### EXAMPLE 3

### a) 2-Acetamido-2-deoxy-1,3,4,6-(tetra-O-benzyl)-α,β-D-glucopyranose

A total of 24.0 g (108.5 mmol) of 2-acetamido-2-deoxy-α,β-D-glucopyranose, dissolved in 500 ml of absolute dimethyl sulfoxide, is added drop by drop at room temperature to a stirred suspension of 20.16 g (700 mmol; 80% in mineral oil) of sodium hydride in 150 ml of dimethyl sulfoxide. Then, it is allowed to stir for 120 more minutes at room temperature, and then 159.5 g (1.26 mol) of benzyl chloride is added in drops. The reaction solution that is thus obtained is subsequently stirred for another 12 hours at room temperature. For working-up, the reaction solution is slowly poured into 1.5 liters of ice water and then exhaustively extracted with diethyl ether. The combined diethyl ether phases are subsequently washed twice with 600 ml of saturated sodium bicarbonate solution each and twice with 800 ml of water each. After the organic phase is dried on sodium sulfate, salt is suctioned out, and the solvent is drawn off in a vacuum. The remaining oily residue is purified on silica gel with use of ethyl acetate/hexane (1:5) as an eluant.

Yield: 48.68 g (73.6% of theory) of the above-mentioned title compound in the form of a viscous and colorless oil

Elementary analysis:

Cld: C, 70.92; H, 6.45; N, 6.89.

Fnd: C, 71.43; H, 6.44; N, 7.02.

### b) 1-O-Benzyl-3,4,6-tri-O-benzyl-2-amino-2-deoxy-α,β-D-glucopyranose

30.0 g (49.2 mmol) of the title compound of Example 3a) is suspended in a mixture of 750 ml of methanol and 215 ml of water and mixed drop by drop at room temperature with a total of 440 ml (49.2 mmol) of a 0.112 molar aqueous perchloric acid solution. After the addition is completed, the reaction solution is stirred for 10 more minutes at room temperature, and the now homogenous reaction solution that is thus obtained is subsequently evaporated to the dry state in a vacuum. By mixing the remaining oily residue with a mixture that consists of equal parts of hexane and dichloromethane, the latter is crystallized. The crystalline reaction product is suctioned off, washed with hexane and dried in a vacuum at room temperature.

Yield: 27.08 g (86% of theory) of the above-mentioned title compound in the form of its perchlorate, which is present as a colorless, crystalline compound.

Melting point: 180.5-181.5° C.

Elementary analysis:

Cld: C, 63.68; H, 5.98; N, 2.19; Cl, 5.54.

Fnd: C, 63.43; H, 6.04; N, 2.02; Cl, 5.71.

### c) 1,3,4,6-Tetra-O-benzyl-2-deoxy-2-[acetyl-(2-amino-N-ethyl-N-perfluorooctylsulfonyl)-amino]-1-α,β-D-glucopyranose

20.8 g (35.6 mmol) of the 2-[N-ethyl-N-perfluorooctylsulfonyl)-aminoacetic acid and 3.60 g (35.6 mmol) of triethylamine are dissolved in 350 ml of dry tetrahydrofuran. After the reaction solution is cooled to −15° C. to −20° C., a solution of 4.92 g (35.6 mmol) of isobutyl chloroformate in 75 ml of dry tetrahydrofuran is slowly added in drops at this temperature while being stirred, whereby the dropwise addition rate is to be selected so that an internal temperature of −10° C. is not exceeded. After a reaction time of 15 minutes at −15° C., a solution of 22.78 g (35.6 mmol) of the perchlorate (title compound of Example 3b) and 3.60 g (35.6 mmol) of triethylamine, in 100 ml of dry tetrahydrofuran, is then slowly added in drops at −20° C. After a reaction time of one hour at −15° C. and two hours at room temperature, the reaction solution is evaporated to the dry state in a vacuum. The remaining residue is taken up in 250 ml of ethyl acetate and washed twice with 100 ml of saturated sodium bicarbonate solution each and once with 200 ml of water. After the organic phase is dried on sodium sulfate, salt is suctioned out, and the ethyl acetate is drawn off in a vacuum. The remaining oily residue is purified on silica gel with use of ethyl-acetate/hexane (1:5) as an eluant.

Yield: 33.3 g (84.6% of theory) of the above-mentioned title compound as a colorless and strongly viscous oil

Elementary analysis:

Cld: C, 49.92; H, 3.92; N, 2.53; F, 29.18; S, 2.90.

Fnd: C, 49.99; H, 4.11; N, 2.69; F, 29.22; S, 3.01.

### d) 2-Deoxy-2-[acetyl-(2-amino-N-ethyl-N-perfluorooctylsulfonyl)-amino]-1-α,β-D-glucopyranose

20.0 g (18.06 mmol) of the title compound of Example 3c) is dissolved in 250 ml of 2-propanol and mixed with 1.5 g of palladium catalyst (10% Pd/C). The reaction solution is hydrogenated for 12 hours at 22° C. and 1 atmosphere of hydrogen pressure. Then, catalyst is filtered out, and the filtrate is evaporated to the dry state. The remaining residue is taken up in 300 ml of dimethyl sulfoxide, and 12.65 g (93.8% of theory) of the above-mentioned title compound is obtained as a colorless and crystalline powder from the product solution that is thus obtained by mixing with 750 ml of a mixture that consists of equal parts of diethyl ether and ethyl acetate after the precipitated solid is suctioned off. The above-mentioned title compound is present as an α/β-anomer mixture, whereby the ratio relative to the two possible anomers was determined at about 1:1.2 by ¹H-NMR-spectroscopic examinations. Accordingly, the title compound is an almost approximately evenly divided α/β-anomer mixture.

Melting point: 132.5-133° C.

Elementary analysis:

Cld: C, 28.97; H, 2.57; N, 3.75; F, 43.27; S, 4.30.

Fnd: C, 29.09; H, 2.56; N, 3.84; F, 43.36; S, 4.42.

### e) Production of a formulation of gadolinium complex III and 2-deoxy-2-[acetyl-(2-amino-N-ethyl-N-perfluorooctylsulfonyl)-amino]-1-α,β-D-glucopyranose

A solution of 4.90 g (6.57 mmol) of the title compound of Example 3d, dissolved in 200 ml of ethanol, is added to 51 ml of a solution of gadolinium complex III (300 mmol/L) that is dissolved in 0.45% sodium chloride solution (pH 7.4/0.25 mg/L CaNa₃DTPA), and it is stirred for 2 hours at 50° C. The solution is evaporated to the dry state in a vacuum, and, with distilled water, the residue yields a total of 153 ml. It is stirred for 10 minutes at 40° C. and filtered through a 0.2 βm filter. The filtrate is decanted into vials. A solution that is thus produced can be used directly for biological experiments. (The concentration is 100 mmol of Gd/L.)

### EXAMPLE 4

### a) 1,2,3,4,6-Penta-O-acetyl-α-D-glucopyranose

Analogously, as described in the synthesis of title compound Ia), the reaction of 100 g (555.0 mmol) of α-D-glucopyranose with a mixture of 1000 ml of absolute pyridine and 1000 ml of acetic acid anhydride after working-up and recrystallization from 95% aqueous ethanol yields 190.6 g (88.0%) of the above-mentioned title compound as a colorless and crystalline compound. By ¹H-NMR-spectroscopic examination of the title compound that is thus obtained, it was possible to determine the α to β-ratio of two possible anomers with ≧98:2. Accordingly, the title compound is the exclusively β-configured anomer.

Melting point: 110.5° C.

Elementary analysis:

Cld: C, 49.21; H, 5.68.

Fnd: C, 49.24; H, 5.68.

### b) 5-(Ethoxycarbonyl)pentyl-2,3,4,6-tetra-O-acetyl-α-D-glucopyranoside

Analogously, as described in the synthesis of the title compound of Example Ib), the reaction of 130.0 g (332.8 mmol) of the title compound of Example 4a) with 55.8 ml (332.8 mmol) of 6-hydroxy-hexanoic acid ethyl ester and 50.6 ml (520 mmol) of tin(IV) chloride in 500 ml of 1,2-dichloroethane after column-chromatographic working-up (eluant: hexane/ethyl acetate 2:1) yields 101.85 g (62.4% of theory) of the above-mentioned title compound as a colorless and viscous oil. According to ¹H-NMR-spectroscopic examination of the title compound, the presence of the β-configuration at the anomeric center could be deduced based on the value of the coupling constant of J_{1,2}=8.8 Hz; moreover, said configuration represents,the sole existing configuration at the anomeric center. It was thus possible to depict the above-mentioned title compound only in the form of the β-configured anomer.

Elementary analysis:

Cld: C, 52.94; H, 6.77.

Fnd: C, 52.77; H, 6.70.

### c) 5-(Carboxy)pentyl-2,3,4,6-tetra-O-benzyl-α-D-glucopyranoside

A stirred suspension of 100.0 g (204.96 mmol) of the title compound of Example 4b) in 150 ml of dioxane is mixed at room temperature and with simultaneous, vigorous stirring in portions with a total of 169.14 g (3.02 mol) of finely powdered potassium hydroxide powder. To increase the stirrability, the reaction mixture is mixed with another 150 ml of dioxane, and the suspension that is thus obtained is subsequently heated to boiling and mixed drop by drop at this temperature with a total of 264 ml (2.218 mol) of benzyl bromide over a period of two hours. After a reaction time of 4 hours at 110° C. followed by 12 hours at room temperature, the reaction mixture is slowly poured into a total of 2.0 liters of ice water for the purpose of working-up, and the water phase is subsequently completely extracted with diethyl ether. After the ether phase that is thus obtained is washed and after the organic phase is subsequently dried on sodium sulfate, salt is suctioned out, and the diethyl ether is drawn off in a vacuum. Excess benzyl bromide is then quantitatively distilled off from the reaction mixture in an oil pump vacuum at an oil bath temperature of 180° C. The remaining oily residue that is thus obtained is purified on silica gel with use of ethyl acetate/hexane (1:10) as an eluant.

Yield: 128.8 g (84.3% of theory) of the above-mentioned title compound in the form of a colorless and extremely viscous oil

Elementary analysis:

Cld: C, 75.68; H, 7.16.

Fnd: C, 75.66; H, 7.23.

### d) 2,3,4,6-Tetra-O-benzyl-1-O-β-D-[6-hexanoic acid-N-(3-oxa-1H,1H,2H,2H,4H,4H,5H,5H-perfluorotridecyl)-amide]-glucopyranose

68.5 g (91.79 mmol) of the acid that is described in Example 4c) and 9.25 g (91.79 mmol) of triethylamine are dissolved in 825 ml of dry tetrahydrofuran. After the reaction solution is cooled to −15° C. to −20° C., a solution of 12.64 g (92.5 mmol) of isobutyl chloroformate in 150 ml of dry tetrahydrofuran is slowly added in drops at this temperature while being stirred, whereby the dropwise addition rate is to be selected such that an internal temperature of −10° C. is not exceeded. After a reaction time of 15 minutes at −15° C., a solution of 46.40 g (91.79 mmol) of 1H,1H,2H,2H-heptadecafluoro-1-(2-aminoethyoxy)-decane and 9.25 g (91.79 mmol) of triethylamine is then slowly added in drops at −20° C. as a solution in 200 ml of dry tetrahydrofuran. After a reaction time of one hour at −15° C., and two hours at room temperature, the reaction solution is evaporated to the dry state in a vacuum. The remaining residue is taken up in 250 ml of ethyl acetate and washed twice with 300 ml of saturated sodium bicarbonate solution each and once with 400 ml of water. After the organic phase is dried on sodium sulfate, salt is suctioned out, and the ethyl acetate is drawn off in a vacuum. The remaining oily residue is purified on silica gel with use of dichloromethane/hexane/2-propanol (10:5:1) as an eluant.

Yield: 104.7 g (92.4% of theory) of the above-mentioned title compound as a colorless and strongly viscous oil.

Elementary analysis:

Cld: C, 57.38; H, 4.98; N, 1.13; F, 26.15.

Fnd: C, 57.27; H, 5.09; N, 1.11; F, 26.08.

### e) 1-O-β-D-[6-Hexanoic acid-N-(3-oxa-1H,1H,2H,2H,4H,4H,5H,5H-perfluorotridecyl)-amide]-glucopyranose

40.0 g (32.38 mmol) of the title compound of Example 4d) is dissolved in 750 ml of 2-propanol and mixed with 2.0 g of palladium catalyst (10% Pd/C). The reaction solution is hydrogenated for 12 hours at 22° C. and 1 atmosphere of hydrogen pressure. Then, catalyst is filtered out, and the filtrate is evaporated to the dry state. The remaining residue is taken up in 300 ml of dimethyl sulfoxide, and 22.05 g (90.2% of theory) of the title compound is obtained as a colorless and crystalline powder with a decomposition melting point of 122-124° C. from the product solution that is thus obtained by mixing with a total of 1000 ml of diethyl ether and subsequent suctioning-off of the precipitated solid.

Elementary analysis:

Cld: C, 36.01; H, 5.92; N, 1.75; F, 40.34.

Fnd: C, 36.07; H, 6.08; N, 1.76; F, 40.66.

### f) Production of a formulation of gadolinium complex IV and 1-O-β-D-[6-hexanoic acid-N-(3-oxa-1H,1H,2H,2H,4H,4H,5H,5H-perfluorotridecyl)-amide]-glucopyranose

20.29 g (25.9 mmol) of the title compound of Example 4e is added to 37 ml of a solution of gadolinium complex IV (300 mmol/L) that is dissolved in 0.45% of aqueous common salt solution (pH 7.4; 0.25 mg/L of CaNa₃DTPA), and, with a 0.9% aqueous common salt solution, it yields a total of 111 ml. It is heated for 2 hours at 60° C. in an ultrasound bath. The solution is cooled to room temperature and set at pH 7.4 with aqueous 2N sodium hydroxide solution. It is filtered through a 0.2 μm filter, and the filtrate is decanted into vials. A solution that is thus produced can be used directly for biological experiments. (The concentration is 100 mmol of Gd/L.)

### EXAMPLE 5

### a) 1-O-(1H,1H,2H,2H-Perfluorodecyl)-(2,3,4,6-tetra-O-acetyl)-α-D-mannopyranose

The reaction of 50 g (128.09 mmol) of the title compound of Example 1a), which is used as a 4:1 mixture relative to the α,β-anomers, with a solution of 75.84 g (128.1 mmol) of 1-hydroxy-1H,1H,2H,2H-perfluorodecane in 150 ml of 1,2-dichloroethane and a total of 19.47 g (166.53 mmol) of tin(IV) chloride, by analogy with the syntheses of the title compounds of Examples 1b) and 4b), results after working-up and column-chromatographic purification (eluant: hexane/ethyl acetate, 2:1) in the formation of 74.2 g (63.4% of theory) of the above-mentioned title compound in the form of a viscous and colorless oil. According to ¹H-NMR-spectroscopic examination of the title compound, the presence of the α-configuration in the anomeric center could be clearly deduced based on the value of the coupling constant of J^{1,2}=1.3 Hz, which is, moreover, the only configuration that is present at the anomerism center, so that accordingly, the above-mentioned title compound could be presented only in the form of the pure α-configured anomer.

Elementary analysis:

Cld: C, 44.65; H, 2.53; F, 35.32.

Fnd: C, 44.77; H, 2.61; F, 35.09.

### b) 1-O-(1H,1H,2H,2H-Perfluorodecyl)-α-D-mannopyranose

25 g (27.33 mmol) of the title compound of Example 5a) is suspended in 400 ml of absolute methanol and mixed at 5° C. with a catalytic amount of sodium methanolate. After a reaction time of 3 hours at room temperature, the thin-layer-chromatographic control (eluant: chloroform/methanol 9:1) of-the course of reaction shows an already quantitative reaction. For the purpose of working-up, the now clear reaction solution is neutralized by mixing with Amberlite IR 120 (H⁺ form)-cation exchange resin, exchanger is suctioned out, and the methanolic filtrate that is thus obtained is drawn off in a vacuum until the material is dry. The crystalline residue that is obtained is purified by recrystallization from ethanol being done twice. According to ¹H-NMR-spectroscopic examination of the title compound, the presence of the α-configuration at the anomeric center could be clearly deduced based on the value of the coupling constant of J_{1,2}=1.0 Hz. This α-configuration is the only configuration that is present at the anomerism center, i.e., the amount of the β-configured anomer of the title compound that can possibly be formed lies below the ¹H-NMR-spectroscopic detection limit. The above-mentioned title compound was accordingly shown only in the form of the pure α-configured anomer.

Yield: 16.2 g (94.6% of theory) of a colorless and crystalline solid

Melting point: 172-174° C. while decomposing

Elementary analysis:

Cld: C, 30.69; H, 2.41; F, 51.57.

Fnd: C, 30.57; H, 2.48; F, 51.65.

### c) Production of a formulation of gadolinium complex II and 1-O-(1H,1H,2H,2H-perfluorodecyl)-α-D-mannopyranose

A solution of 2.01 g (3.21 mmol) of the title compound of Example 5b, dissolved in 200 ml of ethanol, is added to 50 ml of a solution of gadolinium complex II (150 mmol/L) that is dissolved in 0.45% sodium chloride solution (pH 7.4/0.25 mg/L of CaNa₃DTPA), and it is stirred for 2 hours at 50° C. The solution is evaporated to the dry state in a vacuum, and, with distilled water, the residue yields a total of 75 ml. It is stirred for 10 minutes at 40° C. and filtered through a 0.2 μm filter. The filtrate is decanted into vials. A solution that is thus produced can be used directly for biological experiments. (The concentration is 100 mmol of Gd/L.)

### EXAMPLE 6

### a) 1-O-(1H,1H,2H,2H-Perfluorododecyl)-2,3,4,6-tetra-o-acetyl-α-D-mannopyranose

The reaction of 35 g (89.66 mmol) of the title compound of Example 1a), which is used as a 4:1 mixture relative to the α,β-anomer, with a solution of 50.60 g (89.7 mmol) of 1-hydroxy-1H,1H,2H,2H-per-fluorododecane in 100 ml of 1,2-dichloroethane and a total of 13.63 g (16.61 mmol) of tin(IV) chloride, by analogy with the synthesis of the title compounds of Examples 1b), 4b) and 5b), results after working-up and column-chromatrographic purification (eluant: hexane/ethyl acetate=2:1) in the formation of 62.4.9 g (68.7% of~theory) of the above-mentioned title compound in the form of a viscous and colorless oil. According to ¹H-NMR-spectroscopic examination of the title compound, the presence of the α-configuration at the anomeric center could be clearly deduced based on the value~of the coupling constant of J_{1,2}=1.4 Hz, which is, moreover, the only configuration that is present at the anomerism center, so that accordingly the above-mentioned title compound could be depicted only in the form of the pure α-configured anomer.

Elementary analysis:

Cld: C, 42.62; H, 2.28; F, 39.32.

Fnd: C, 42.55; H, 2.38; F, 39.40.

### b) 1-O-(1H,1H,2H,2H-Perfluorododecyl)-α-D-mannopyranose

25 g (24.64 mmol) of the title compound of Example 6a) is suspended in 400 ml of absolute methanol and mixed at 5° C. with a catalytic amount of sodium methanolate. After a reaction time of 3 hours at room temperature, the thin-layer-chromatographic control (eluant: chloroform/methanol=9:1) of the course of reaction indicates an almost quantitative reaction. For the purpose of working-up, the now clear reaction solution is neutralized by mixing with Amberlite IR 120 (H⁺ form)-cation exchange resin, exchanger is suctioned out, and the methanolic filtrate that is thus obtained is drawn off in a vacuum until the material is dry. The crystalline residue that is obtained is purified by recrystallization, twice, of a mixture of 2-propanol/ethanol (1:1). According to ¹H-NMR-spectroscopic examination of the title compound, the presence of the α-configuration at the anomeric center could be clearly deduced based on the value of the coupling constant of J_{1,2}=0.9 Hz. This α-configuration is the only configuration that is present at the anomerism center, i.e., the amount of the β-configured anomer of the title compound that can possibly be formed lies below the ¹H-NMR spectroscopic detection limit. The above-mentioned title compound was accordingly depicted only in the form of the pure α-configured anomer.

Yield: 16.96 g (90.8% of theory) of a colorless and crystalline solid

Melting point: 187-188° while decomposing.

Elementary analysis:

Cld: C, 29.77; H, 2.08; F, 54.93.

Fnd: C, 29.70; H, 2.28; F, 54.83.

### c) Production of a formulation of gadolinium complex V and 1-O-(1H,1H,2H,2H-perfluorododecyl)-α-D-mannopyranose

1.70 g (2.34 mmol) of the title compound of Example 6b is added to 52 ml of a solution of gadolinium complex V (180 mmol/L) that is dissolved in 0.45% of aqueous sodium chloride solution, and it is heated for 10 minutes in the microwave. The solution is cooled to room temperature, filtered through a 0.2 μm filter, and the filtrate is decanted into vials. A solution that is thus produced can be used directly for biological experiments. (The concentration is 180 mmol of Gd/L.)

### EXAMPLE 7

### a) 2,3,4,6-Tetra-O-acetyl)-1-O-α-D-[3,6,9-trioxa-(C₁₂-C₁₉-heptadecafluoro)-nonadecyl]-mannopyranose

The reaction of 20 g (51.23 mmol) of the title compound of Example 1a), which is used as a 4:1 mixture relative to the α,β-anomers, with a solution of 30.54 g (51.23 mmol) of 1-hydroxy-tris-(1H,1H,2H,2H-0)-1H,1H,2H,2H-perfluorodecane in 100 ml of 1,2-dichloroethane and a total of 5.98 g (51.23 mmol) of tin(IV) chloride, by analogy with the syntheses of the title compounds of Examples 1b), 4b) and 5b), results after working-up and column-chromatographic purification (eluant: hexane/ethyl acetate=1:1) in the formation of 34.22 g (72.1% of theory) of the above-mentioned title compound in the form of a viscous and colorless oil. According to ¹H-NMR-spectroscopic examination of the title compound, the presence of the α-configuration at the anomeric center could be deduced based on the value of the coupling constant of J_{1,2}=1.1 Hz, which, moreover, is the only configuration that is present at the anomerism center, so that accordingly the above-mentioned title compound could be depicted only in the form of the pure α-configured anomer.

Elementary analysis:

Cld: C, 38.89; H, 3.81; F, 34.86.

Fnd: C, 39.02; H, 3.77; F, 34.90.

### b) 1-O-α-D-[3,6,9-Trioxa-(C₁₂-C₁₉-heptadecafluoro)-nonadecyl]-mannopyranose

20 g (21.58 mmol) of the title compound of Example 7a) is suspended in 350 ml of absolute methanol and mixed at 5° C. with a catalytic amount of sodium methanolate. After a reaction time of 3 hours at room temperature, the thin-layer-chromatographic control (eluant: chloroform/methanol=6:1) of the course of the reaction indicated already quantitative reaction. For working-up, the now clear reaction solution is neutralized by mixing with Amberlite IR 120 (H⁺ form)-cation exchange resin, exchanger is suctioned off, and the methanolic filtrate that is thus obtained is drawn off in a vacuum until the material is dry. The crystalline residue that is obtained is purified by recrystallization being done twice from a mixture of ethyl acetate/2-propanol/ethanol (1:0.5:1). According to ¹H-NMR-spectroscopic examination of the title compound, the presence of the α-configuration at the anomeric center could be deduced based on the value of the coupling constant to J_{1,2}=1.0 Hz. This α-configuration is the only configuration that is present at the anomerism center, i.e., the amount of the β-configured anomer of the title compound that can possibly be formed lies below the ¹H-NMR-spectroscopic detection limit. The above-mentioned title compound was depicted accordingly only in the form of the pure α-configured anomers.

Yield: 15.20 g (92.9% of theory) of a colorless, crystalline solid

Melting point: 141° C.

Elementary analysis:

Cld: C, 34.84; H, 3.59; F, 42.58.

Fnd: C, 34.72; H, 3.66; F, 42.67.

### c) Production of a formulation of gadolinium complex VIII and 1-O-α-D-[3,6,9-trioxa-(C₁₂-C₁₉-heptadecafluoro)-nonadecyl]-mannopyranose

3.71 g (4.89 mmol) of the title compound of Example 7b is added to 38 ml of a solution of gadolinium complex VIII (300 mmol/L) that is dissolved in 0.45% of aqueous common salt solution (pH 7.4: 0.25 mg/L of CaNa₃DTPA), and, with a 0.9% aqueous common salt solution, it yields a total of 114 ml. It is heated for 2 hours at 60° C. in an ultrasound bath. The solution is cooled to room temperature and set at pH 7.4 with aqueous 2N sodium hydroxide solution. It is filtered through a 0.2 Am filter, and the filtrate is decanted into vials. A solution that is thus produced can be used directly for biological experiments. (The concentration is 100 mmol of Gd/L.)

### EXAMPLE 8

### a) 2,3,4,6-Tetra-O-acetyl-1-α-D-[3-thiopropionic acid-N-(3-oxa-1H,1H,2H,2H,4H,4H,5H,5H-perfluorotridecyl)-amide]-mannopyranose

25.0 g (57.28 mmol) [for production according to Ponpipom, Mitree, M.; Bugianesi, Robert L.; Robbins, James, C.; Doebber, T. W.; Shen, T. Y.; J. Med. Chem.; 24; 12; 1981; 1388-1395] of 3-(tetra-O-acetyl-α-D-mannopyranosyl-mercapto)-propionic acid and 5.77 g (57.28 mmol) of triethylamine are dissolved in 500 ml of dry tetrahydrofuran. After the reaction solution is cooled to −15° C. to −20° C., a solution of 7.82 g (57.28 mmol) of isobutyl chloroformate in 100 ml of dry tetrahydrofuran is slowly added in drops at this temperature while being stirred, whereby the rate of addition by drops is to be selected so that an internal temperature of −10° C. is not exceeded. After a reaction time of 15 minutes at −15° C., a solution of 29.05 g (57.28 mmol) of 1H,1H,2H,2H-heptadecafluoro-1-(2-aminoethyoxy)-decane and 5.77 g (57.28 mmol) of triethylamine is subsequently slowly added in drops as a solution in 200 ml of dry tetrahydrofuran at −20° C. After a reaction time of one hour at −15° C. and for two hours at room temperature, the reaction solution is evaporated to the dry state in a vacuum. The remaining residue is taken up in 250 ml of ethyl acetate, and washed twice with 200 ml of saturated sodium bicarbonate solution each and once with 300 ml of water. After the organic phase is dried on sodium sulfate, salt is suctioned out, and the ethyl acetate is drawn off in a vacuum. The remaining oily residue is purified as an eluant on silica gel with use of dichloromethane/hexane/2-propanol (8:5:1).

Yield: 44.90 g (84.7% of theory) of the above-mentioned title compound as a colorless and strongly viscous oil.

Elementary analysis:

Cld: C, 37.63; H, 3.48; N, 1.51; S, 3.46; F, 34.89.

Fnd: C, 37.77; H, 3.37; N, 1.61; S, 3.57; F, 35.21.

### b) 1-α-D-[3-Thiopropionic acid-N-(3-oxa-1H,1H,2H,2H,4H,4H,5H,5H-perfluorotridecyl)-amide]-mannopyranose

30 g (32.41 mmol) of the title compound of Example 8a) is suspended in 400 ml of absolute methanol and mixed at 5° C. with a catalytic amount of sodium methanolate. After a reaction time of 3 hours at room temperature, the thin-layer-chromatic control (eluant: chloroform/methanol=9:1) of the course of the reaction indicates an already quantitative reaction. For working-up, the now clear reaction solution is neutralized by mixing with Amberlite IR 120 (H⁺ form)-cation exchange resin, exchanger is suctioned out, and the methanolic filtrate that is thus obtained is drawn off in a vacuum until the material is dry. The crystalline residue that is obtained is purified by recrystallization from a mixture that consists of ethyl acetate/methanol (0.5:1). According to ¹H-NMR-spectroscopic examination of the title compound, the presence of the α-configuration at the anomeric center could be deduced based on the value of the coupling constant of J_{1,2}=1.1 Hz. This α-configuration is the only configuration that is present at the anomerism center, i.e., the amount of the β-configured anomer of the title compound that can possibly be formed lies below the ¹H-NMR-spectroscopic detection limit. The above-mentioned title compound was depicted accordingly only in the form of the pure α-configured anomers.

Yield: 23.76 g (96.8% of theory) of a colorless and crystalline solid

Melting point: 113-114.5° C.

Elementary analysis:

Cld: C, 33.30; H, 3.19; N, 1.85; S, 4.23; F, 42.64.

Fnd: C, 33.21; H, 3.26; N, 1.96; S, 4.08; F, 42.77.

### c) Production of a formulation of gadolinium complex VI and 1-α-D-[3-thio-propionic acid-N-(3-oxa-1H,1H,2H,2H,4H,4H,5H,5H-perfluorotridecyl)-amide]-mannopyranose

A solution of 27.41 g (36.19 mmol) of the title compound of Example 8b, dissolved in 200 ml of ethanol, is added to 47 ml of a solution of gadolinium complex VI (330 mmol/L), dissolved in 0.45% sodium chloride solution (pH 7.4/0.25 mg/L of CaNa₃DTPA), and it is stirred for 2 hours at 50° C. The solution is evaporated to the dry state in a vacuum, and, with distilled water, the residue yields a total of 155 ml. It is stirred for 10 minutes at 40° C. and filtered through a 0.2 μm filter. The filtrate is decanted into vials. A solution that is thus produced can be used directly for biological experiments. (The concentration is 100 mmol of Gd/L.)

### EXAMPLE 9

### a) 2,3,4,6-Tetra-O-acetyl-1-β-D-[3,6,9-trioxa-(C₁₂-C₁₉-heptadecafluoro)-nonadecyl]-glucopyranosyluronic acid

250 ml of anhydrous acetonitrile is dissolved to form a stirred solution of 20.2 g (50.85 mmol) of methyl (1-bromo-2,3,4-tri-O-acety-α-D-glucopyranosid)uronate [For production according to: Pelzer; Hoppe-Seyler's Z. Physiol. Chem.; 314; 1949; 234, 237 and Goebel; Babers; J. Biol. Chem.; 111; 1935; 347, 350 and Bollenback et al.; J. Amer. Chem. Soc.; 77; 1955; 3310, 3313] and 60.64 g (101.7 mmol) of 3,6,9-trioxa-(C₁₂-C₁₉ heptadecafluoro)-nonadecan-1-ol, and it is mixed at room temperature with 13.0 g of freshly precipitated silver oxide. After a reaction time of 12 hours at room temperature, the insoluble silver salts are filtered out, the salts are rewashed well with dichloromethane, and the filtrate that is thus obtained is drawn off in a vacuum until the material is dry. The remaining residue is purified by column chromatography (eluant: hexane/ethyl acetate=3:1).

Yield: 22.99 g (53.3% of theory) of the above-mentioned title compound as a colorless, highly viscous oil

Elementary analysis:

Cld: C, 41.05; H, 3.92; F, 38.06.

Fnd: C, 41.20; H, 3.76; F, 38.22.

### b) 1-O-β-D-(3,6,9-Trioxa-(C₁₂-C₁₉-heptadecafluoro)-nonadecyl]-glucopyranosyluronic acid

10.0 g (11.78 mmol) of the title compound of Example 9a) is suspended in 200 ml of a mixture that consists of methanol and 0.5 molar sodium hydroxide solution at a ratio of 2:1 while being stirred at room temperature. After a reaction time of 12 hours at room temperature, the now clear reaction mixture is neutralized for working-up by mixing with Amberlite IR 120 (H⁺ from)-cation exchange resin, exchanger is suctioned out, and the methanolic-aqueous filtrate that is thus obtained is drawn off in a vacuum until the material is dry. The crystalline residue that is obtained is purified by recrystallization from a mixture of ethyl acetate/methanol (0.25:1). According to ¹H-NMR-spectroscopic examination of the title compound, the presence of the β-configuration at the anomeric center could be deduced based on the value of the coupling constant of J_{1,1}=9.2 Hz. This β-configuration is the only configuration that is present at the anomerism center, i.e., the amount of the β-configured anomer of the title compound that can possibly be formed lies below the ¹H-NMR-spectroscopic detection limit. The above-mentioned title compound was depicted accordingly only in the form of the pure β-configured anomer.

Melting point: 78.5° C.

Elementary analysis:

Cld: C, 34.21; H, 3.26; F, 41.81.

Fnd: C, 34.38; H, 3.26; F, 41.90.

### c) Production of a formulation that consists of gadolinium complex I and 1-O-β-D-[3,6,9-trioxa-(C₁₂-C₁₉-heptadecafluoro)-nonadecyl]-glucopyranosyluronic acid

19.18 g (24.83 mmol) of the title compound of Example 9b is added to 38 ml of a solution of the gadolinium complex (280 mmol/L), dissolved in 0.45% aqueous common salt solution (pH 7.4; 0.25 mg/L of CaNa₃DTPA), and, with a 0.9% aqueous common salt solution, it yields a total of 53.2 ml. It is heated for 2 hours at 60° C. in an ultrasound bath. The solution is cooled to room temperature and set at pH 7.4 with aqueous 2N sodium hydroxide solution. It is filtered through a 0.2 μm filter, and the filtrate is decanted into vials. A solution that is thus produced can be used directly for biological experiments. (The concentration is 200 mmol of Gd/L.)

### EXAMPLE 10

### a) 6-(2-Oxa-1H,1H,3H,3H,4H,4H-perfluorodecyl)-O¹,O²,O³,O⁴-diisopropylidene-α-D-galactopyranose

A total of 12.15 g (46.66 mmol) of O¹,O²,O³,O⁴-diisopropylidene-α-galactopyranose [production according to: Levene; Meyer; J. Biol. Chem.; 64; 1925; 473 and McCreath; Smith; J. Chem. Soc.; 1939; 387, 389 and Freudenberg; Hixon; Chem. Ber.; 56; 1923; 2119, 2122], dissolved in 200 ml of absolute dimethylformamide, is added drop by drop at room temperature to a stirred suspension of 2.01 g (70.0 mmol/80% in mineral oil) of sodium hydride in 25 ml of dimethylformamide. Then, it is allowed to stir for 120 more minutes at room temperature, and a total of 30.09 g (48.0 mmol) of 1-bromo-1H,1H,2H,2H-perfluorododecane, dissolved in 150 ml of absolute dimethylformamide, is subsequently slowly added in drops. The reaction solution that is thus obtained is subsequently stirred for another 12 hours at room temperature. For working-up, the reaction solution is slowly poured into 1 liter of ice water and then exhaustively extracted with diethyl ether. The combined organic phases are subsequently washed twice with 200 ml of saturated sodium bicarbonate solution each and twice with 200 ml of water each. After the organic phase is dried on sodium sulfate, salt is suctioned out, and the solvent is drawn off in a vacuum. The remaining oily residue is purified on silica gel with use of ethyl acetate/hexane (1:10) as an eluant.

Yield: 29.8 g (79.3% of theory) of the above-mentioned title compound in the form of a viscous, colorless oil

Elementary analysis:

Cld: C, 35.75; H, 2.87; F, 49.47.

Fnd: C, 35.64; H, 2.98; F, 49.54.

### b) 6-(2-Oxa-1H,1H,3H,3H,4H,4H-perfluorodecyl)-α-D-galactopyranose

20 g (24.8 mmol) of the title compound of Example 10a) is mixed with 300 ml of a 1% aqueous sulfuric acid solution and stirred for 3 hours at 80° C. After cooling to room temperature, it is neutralized by mixing with aqueous barium hydroxide solution, and precipitated barium sulfate is subsequently filtered out, and the clear aqueous product solution that is thus obtained is freeze-dried. By ¹H-NMR-spectroscopic examination of the title compound, the presence of the two possible configurations at the anomeric center could be shown clearly, whereby this α/β-configuration ratio was determined according to ¹H-NMR-spectroscopic examination with 1:1.4 (α:β) at the anomerism center. The above-mentioned title compound was accordingly isolated only in the form of the 1:1.4 (α:β)-anomer mixture, i.e., an anomeric separation was eliminated.

Yield: 15.28 g (98.4% of theory) of the above-mentioned title compound as a colorless lyophilizate

Elementary analysis (relative to anhydrous substance):

Cld: C, 35.75; H, 2.87; F, 49.47.

Fnd: C, 35.64; H, 2.98; F, 49.54.

### c) Production of a formulation of gadolinium complex VII and 6-(2-oxa-1H,1H,3H,3H,4H,4H-perfluorodecyl)-α-D-galactopyranose

A solution of 1.68 g (2.69 mmol) of the title compound of Example 10b, dissolved in 200 ml of ethanol, is added to 43 ml of a solution of gadolinium complex VII (250 mmol/L), dissolved in 0.45% sodium chloride solution (pH 7.4/0.25 mg/L of CaNa₃DTPA), and it is stirred for 2 hours at 50° C. The solution is evaporated to the dry state in a vacuum, and, with distilled water, the residue yields a total of 107.5 ml. It is stirred for 10 minutes at 40° C. and filtered through a 0.2 μm filter. The filtrate is decanted into vials. A solution that is thus produced can be used directly for biological experiments. (The concentration is 100 mmol of Gd/L-.)

### EXAMPLE 11

### a) 1-O-α-D-[(1-Perfluorooctylsulfonylpiperazine-4-carbonyl)-methyl]-mannopyranose

30 g (52.8 mmol) of 1-perfluorooctylsulfonylpiperazine (production described in DE 196 03 033) and 31.73 g (53 mmol) of 2,3,4,6-tetra-O-benzyl-α-D-carboxymethyl-mannopyranose (production described in DE 197 28 954) are dissolved in 300 ml of tetrahydrofuran. At 0° C., 24.73 g (100 mmol) of EEDQ (=1,2-dihydro-2-ethoxy-quinoline-1-carboxylic acid ethyl ester) is added, and it is stirred for 3 hours at 0° C., then for 6 hours at room temperature. The solution is evaporated to the dry state in a vacuum, and the residue is purified by flash chromatography on silica gel (mobile solvent: hexane/ethyl acetate=10:1). The product-containing fractions are evaporated to the dry state, the residue is dissolved in a mixture of 200 ml of methanol/150 ml of dichloromethane and hydrogenated for 8 hours on palladium/carbon (10% Pd/C 2 g). Hydrogenating catalyst is filtered out, and the filtrate is evaporated to the dry state. The residue is recrystallized from acetone/diethyl ether.

Yield: 30.39 g (73% of theory) of a waxy, colorless solid

Elementary analysis:

Cld: C, 30.47; H, 2.68; F, 40.96; N, 3.55; S, 4.07.

Fnd: C, 30.61; H, 2.75; F, 41.10; N, 3.46; S, 4.12.

### b) Production of a formulation that consists of gadolinium complex I-and 1-O-α-D-C(1-perfluorooctylsulfonylpiperazine-4-carbonyl-)-methyl]-mannopyranose

4.71 g (5.97 mmol) of the title compound of Example 11a is added to 32 ml of a solution of gadolinium complex I (280 mmol/L), dissolved in 0.45% aqueous common salt solution (pH 7.4; 0.25 mg/L of CaNa₃DTPA), and, with a 0.9% aqueous common salt solution, it yields a total of 55 ml. It is heated for 2 hours at 60° C. in an ultrasound bath. The solution is cooled to room temperature and set at pH 7.4 with aqueous 2N sodium hydroxide solution. It is filtered through a 0.2 μm filter, and the filtrate is decanted into vials. A solution that is thus produced can be used directly for biological experiments. (The concentration is 200 mmol of Gd/L.)

### EXAMPLE 12

### a) 3-Oxa-2H,2H,4H,4H,5H,5H-perfluorotridecanoic acid, sodium salt

20 g (38.3 mmol) of 3-oxa-2H,2H,4H,4H,5H,5H-perfluorotridecanoic acid (production described in DE 196 03 033) is dissolved in 300 ml of ethanol, and 7.7 ml of 5N aqueous sodium hydroxide solution is added. It is evaporated to the dry state, and the residue is dried in a vacuum-drying oven (8 hours at 60° C.).

Yield: 20.85 g (quantitative) of a colorless, crystalline powder

Elementary analysis:

Cld: C, 26.49; H, 1.11; F, 59.35; Na, 4.22.

Fnd: C, 26.60; H, 1.19; F, 59.47; Na, 4.30.

### b) Production of a formulation that consists of gadolinium complex I and 3-oxa-2H,2H,4H,4H,5H,5H-perfluorotridecanoic acid, sodium salt

2.09 g (3.84 mmol) of the title compound of Example 12a is added to 32 ml of a solution of gadolinium complex I (280 mmol/L), dissolved in 0.45% aqueous common salt solution (pH 7.4; 0.25 mg/L of CaNa₃DTPA), and, with a 0.9% aqueous common salt solution, it yields a total of 90 ml. It is heated for 2 hours at 60° C. in an ultrasound bath. The solution is cooled to room temperature and set at pH 7.4 with aqueous 2N sodium hydroxide solution. It is filtered through a 0.2 μm filter, and the filtrate is decanted into vials. A solution that is thus produced can be used directly for biological experiments. (The concentration is 100 mmol of Gd/L.)

### c) Production of a formulation that consists of gadolinium complex I and 3-oxa-2H,2H,4H,4H,5H,5H-perfluorotridecanoic acid, sodium salt

1.00 g (1.84 mmol) of the title compound of Example 12a is added to 32 ml of a solution of gadolinium complex I (280 mmol/L) that is dissolved in 0.45% aqueous common salt solution (pH 7.4; 0.25 mg/L of CaNa₃DTPA), and, with a 0.9% aqueous common salt solution, it yields a total of 90 ml. It is heated for 2 hours at 60° C. in an ultrasound bath. The solution is cooled to room temperature and set at pH 7.4 with aqueous 2N sodium hydroxide solution. It is filtered through a 0.2 μm filter, and the filtrate is decanted into vials. A solution that is thus produced can be used directly for biological experiments. (The concentration is 100 mmol of Gd/L.)

### d) Production of a formulation that consists of gadolinium complex I and 3-oxa-2H,2H,4H,4H,5H,5H-perfluorotridecanoic acid, sodium salt

0.54 g (1.0 mmol) of the title compound of Example 12a is added to 32 ml of a solution of gadolinium complex I (280 mmol/L), dissolved in 0.45% aqueous common salt solution (pH 7.4: 0.25 mg/L of CaNa₃DTPA), and, with a 0.9% aqueous common salt solution, it yields a total of 90 ml. It is heated for 2 hours at 60° C. in an ultrasound bath. The solution is cooled to room temperature and set at pH 7.4 with aqueous 2N sodium hydroxide solution. It is filtered through a 0.2 Am filter, and the filtrate is decanted into vials. A solution that is thus produced can be used directly for biological experiments. (The concentration is 100 mmol of Gd/L.)

### EXAMPLE 13

### a) 1-Perfluorooctylsulfonyl-4-(3,6,9,12,15-pentaoxahexadecanoyl)-piperazine

20 g (35.2 mmol) of perfluoroacetylsulfonylpiperazine (see Example 11a) is dissolved in 300 ml of dichloromethane, and 5.06 g (50 mmol) of triethylamine is added. It is cooled to 0° C., and 14.24 g (50 mmol) of 3,6,9,12,15-pentaoxahexanoic acid chloride is added in drops within 20 minutes and-stirred for 3 hours at 0° C. 400 ml of 5% aqueous hydrochloric acid is added and thoroughly stirred. The organic phase is separated, dried on magnesium sulfate and evaporated to the dry state in a vacuum. The residue is chromatographed on silica gel (mobile solvent=dichloromethane/methanol: 15:1).

Yield: 26.44 (92% of theory) of a waxy solid

Elementary analysis:

Cld: C, 33.83; H, 3.58; N, 3.43; F, 39.55; S, 3.93.

Fnd: C, 33.96; H, 3.66; N, 3.50; F, 39.67; S, 3.82.

### b) Production of a formulation that consists of gadolinium complex I and 1-perfluorooctyl-sulfonyl-4-(3,6,9,12,15-pentaoxahexadecanoyl)-piperazine

4.61 g (5.64 mmol) of the title compound of Example 13a is added to 47 ml of a solution of gadolinium complex I (280 mmol/L), dissolved in 0.45% aqueous common salt solution (pH 7.4; 0.25 mg/L of CaNa₃DTPA), and, with a 0.9% aqueous common salt solution, it yields a total of 66 ml. It is heated for 2 hours at 60° C. in an ultrasound bath. The solution is cooled to room temperature and set at pH 7.4 with aqueous 2N sodium hydroxide solution. It is filtered through a 0.2 μm filter, and the filtrate is decanted into vials. A solution that is thus produced can be used directly for biological experiments. (The concentration is 200 mmol of Gd/L.)

### EXAMPLE 14

### a) 1H,1H,2H,2H-Perfluorodecyl-p-toluenesulfonic acid ester

20 g (43.1 mmol) of 1H,1H,2H,2H-perfluorodecanol is dissolved in 200 ml of pyridine, and 9.53 g (50 mmol) of p-toluenesulfonic acid chloride is added in portions at 0° C. It is stirred for 5 hours at room temperature. The solution is poured into 1000 ml of ice water and stirred for 10 minutes. The precipitate is filtered off, washed with a lot of water and then recrystallized from acetone.

Yield: 22.04 g (97% of theory) of a colorless, crystalline solid

Elementary analysis:

Cld: C, 22.78; H, 0.76; F, 61.26; S, 6.08.

Fnd: C, 22.89; H, 0.70; F, 61.39; S, 6.15.

### b) C₁₈-C₂₅-Heptadeca-fluoro-3,6,9,12,15-pentaoxa-pentacosan-1-ol

20 g (37.94 mmol) of the title compound of Example 14a, 35.74 g (150 mmol) of pentaethylene glycol and 1 g of 18-crown ether-6 are dissolved in 300 ml of tetrahydrofuran, and 10.1 g (180 mmol) of finely powdered potassium hydroxide is added. It is stirred for 10 hours at room temperature. Solid is filtered out, and the filtrate is evaporated to the dry state in a vacuum. The residue is chromatographed on silica gel (mobile solvent: dichloromethane/methanol=15:1).

Yield: 5.45 g (21% of theory) of a colorless, viscous oil

Elementary analysis:

Cld: C, 35.10; H, 3.68; F, 47.19.

Fnd: C, 35.22; H, 3.77; F, 47.10.

### c) Production of a formulation that consists of gadolinium complex IX and C₁₈-C₂₅ hepta-deca-fluoro-3,6,9,12,15-pentaoxa-pentacosan-1-ol,

44.98 g (65.72 mmol) of the title compound of Example 14b is added to 53 ml of a solution of gadolinium complex IX (310 mmol/L), dissolved in 0.45% of aqueous sodium chloride solution), and it is heated for 10 minutes in the microwave. The solution is cooled to room temperature, filtered through a 0.2 μm filter, and the filtrate is decanted into vials. A solution that is thus produced can be used directly for biological experiments. (The concentration is 310 mmol of Gd/L.)

### EXAMPLE 15

### a) N,N-Bis(8-hydroxy-3,6-dioxa-octyl)-perfluorooctylsulfonic acid amide

15 g (29.23 mmol) of perfluorooctylsulfonic acid amide and 22.16 g (87.7 ml) of 9-(tetrahydropyran-2-yl)-3,6,9-trioxa-nonyl chloride are dissolved in 200 ml of acetonitrile. 41.46 g (300 mmol) of potassium carbonate and 1 g (6 mmol) of potassium iodide are added and refluxed for 10 hours. The solid is filtered off, and the filtrate is evaporated to the dry state in a vacuum. The residue is dissolved in 400 ml of ethanol, and 30 ml of 10% aqueous hydrochloric acid is added. It is stirred for 2 hours at room temperature. It is set at pH 7 with sodium hydroxide solution, and the solution is concentrated by evaporation in a vacuum. The residue is chromatographed on silica gel (mobile solvent: dichloromethane/methanol=10:1).

Yield: 11.38 g (51% of theory) of a colorless, viscous oil

Elementary analysis:

Cld: C, 31.46; H, 3.43; N, 1.83; F, 42.30; S, 4.20.

Fnd: C, 31.59; H, 3.50; N, 1.90; F, 42.46; S, 4.08.

### b) Production of a formulation that consists of gadolinium complex I and N,N-bis(8-hydroxy-3,6-dioxa-octyl)-perfluorooctylsulfonic acid amide

7.91 g (10.36 mmol) of the title compound of Example 15a is added to 37 ml of a solution of gadolinium complex I (280 mmol/L), dissolved in 0.45% aqueous common salt solution (pH 7.4: 0.25 mg/L of CaNa₃DTPA), and, with a 0.9% aqueous common salt solution, it yields a total of 104 ml. It is heated for 2 hours at 60° C. in an ultrasound bath. The solution is cooled to room temperature and set at pH 7.4 with an aqueous 2N sodium hydroxide solution. It is filtered through a 0.2 μm filter, and the filtrate is decanted into vials. A solution that is thus produced can be used directly for biological experiments. (The concentration is 100 mmol of Gd/L.)

### EXAMPLE 16

### a) N,N-Bis(t-butyloxycarbonylmethyl)-perfluorooctylsulfonic acid amide

20 g (38.97 mmol) of perfluorooctylsulfonic acid amide and 20.73 g (150 mol) of potassium carbonate are suspended in 200 ml of acetone, and 17.56 g (90 mmol) of bromoacetic acid-tert-butyl ester is added. It is refluxed for 3 hours. The solid is filtered off, and the filtrate is evaporated to the dry state in a vacuum. The residue is chromatographed on silica gel (mobile solvent: n-hexane/ethyl acetate=10:1).

Yield: 23.53 g (83% of theory) of a colorless, waxy solid

Elementary analysis:

Cld: C, 33.02; H, 3.05; F, 44.40; N, 1.93; S, 4.41.

Fnd: C, 33.19; H, 3.11; F, 44.30; N, 1.99; S, 4.32.

### b) N,N-Bis(carboxymethyl)-perfluorooctylsulfonic acid amide, disodium salt

23 g (31.62 mmol) of the title compound of Example 16a is dissolved in 300 ml of trifluoroacetic acid and stirred for 5 hours at room temperature. It is evaporated to the dry state in a vacuum, and the residue is recrystallized from acetone. The crystals are in a vacuum (dried at 50° C./hours)

Yield: 17.7 g (91% of theory) of a colorless, crystalline powder

17 g (27.63 mmol) of the dioic acid that is thus obtained is dissolved in 100 ml of water/300 ml of ethanol, and 9.2 ml of 3N aqueous sodium hydroxide solution is added. It is stirred for 20 minutes at room temperature and then evaporated to the dry state in a vacuum. The residue is dried in a vacuum (60° C./8 hours)

Yield: 18.2 g of colorless, crystalline powder

Elementary analysis:

Cld: C, 21.87; H, 0.61; N, 2.12; F, 49.00; S, 4.86; Na, 6.98.

Fnd: C, 22.00; H, 0.70; N, 2.20; F, 49.17; S, 4.93; Na, 7.10.

### c) Production of a formulation that consists of gadolinium complex II and N,N-bis(carboxy-methyl)-perfluorooctylsulfonic acid amide, disodium salt

2.89 g (4.39 mmol) of the title compound of Example 16b is added to 41 ml of a solution of gadolinium complex II (250 mmol/L), dissolved in 0.45% of aqueous common salt solution (pH 7.4; 0.25 mg/L of CaNa₃DTPA), and, with a 0.9% aqueous common salt solution, it yields a total of 52 ml. It is heated for 2 hours at 60° C. in an ultrasound bath. The solution is cooled to room temperature and set at pH 7.4 with aqueous 2N sodium hydroxide solution. It is filtered through a 0.2 μm filter, and the filtrate is decanted into vials. A solution that is thus produced can be used directly for biological experiments. (The concentration is 200 mmol of Gd/L.)

### EXAMPLE 17

### a) 1H,1H,2H,2H-Perfluorododecyl-sulfuric acid monoester, sodium salt

10 g (17.73 mmol) of 1H,1H,2H,2H-perfluorododecanol is dissolved in 300 ml of chloroform, and 2.82 g (17.73 mmol) of sulfur trioxide-pyridine-complex is added at 0° C. It is stirred for one hour at 0° C. and then evaporated to the dry state in a vacuum. The residue is dissolved in 300 ml of ethanol and mixed with 17.8 ml of 1N aqueous sodium hydroxide solution. The solution is evaporated to the dry state, and the residue is dried in a vacuum (60° C./2 hours).

Yield: 11.81 g (quantitative)

Elementary analysis:

Cld: C, 21.64; H, 0.61; F, 59.89; Na, 3.45; S, 4.81.

Fnd: C, 21.70; H, 0.72; F, 60.00; Na, 3.57; S, 4.92.

### b) Production of a formulation that consists of gadolinium complex V and 1H,1H,2H,2H-perfluorododecyl-sulfuric acid monoester, sodium salt

4.90 g (7.35 mmol) of the title compound of Example 17a is added to 38 ml of a solution of gadolinium complex V (290 mmol/L), dissolved in 0.45% aqueous sodium chloride solution), and it is heated for 10 minutes in the microwave. The solution is cooled to room temperature, filtered through a 0.2 μm filter, and the filtrate is decanted into vials. A solution that is thus produced can be used directly for biological experiments. (The concentration is 290 mmol of Gd/L.)

### EXAMPLE 18

### a) 2H,2H,4H,4H,5H,5H-3-oxa-perfluoropentadecanoic acid, sodium salt

10 g (16.07 mmol) of 2H,2H,4H,4H,5H,5H-3-oxa-perfluoropentadecanoic acid is dissolved in 300 ml of ethanol and mixed with 16.1 ml of 1N aqueous sodium hydroxide solution. The solution is evaporated to the dry state, and the residue is dried in a vacuum (60° C./2 hours)

Yield: 10.35 g (quantitative) of a colorless, amorphous powder

Elementary analysis:

Cld: C, 26.10; H, 0.94; F, 61.94; Na, 3.57.

Fnd: C, 26.22; H, 1.00; F, 62.05; Na, 3.66.

### b) Production of a formulation that consists of gadolinium complex VI and 2H,2H,4H,4H,5H,5H-3-oxa-perfluoropentadecanoic acid, sodium salt

A solution of 3.36 g (5.21 mmol) of the title compound of Example 18a, dissolved in 200 ml of ethanol, is added to 45 ml of a solution of gadolinium complex VI (270 mmol/L), dissolved in 0.45% sodium chloride solution (pH 7.4/0.25 mg/L of CaNa₃DTPA), and it is stirred for 2 hours at 50° C. The solution is evaporated to the dry state in a vacuum, and, with distilled water, the residue yields a total of 122 ml. It is stirred for 10 minutes at 40° C. and filtered through a 0.2 μm filter. The filtrate is decanted into vials. A solution that is thus produced can be used directly for biological experiments. (The concentration is 100 mmol of Gd/L.)

### EXAMPLE 19

### a) Ethylenediamine-N,N-tetraacetic acid-N-(1H,1H,2H,2H,4H,4H,5H,5H-3-oxa-perfluorotridecyl)-monoamide

10.14 g (20 mmol) of 1H,1H,2H,2H,4H,4H,5H,5H-3-oxa-perfluorotridecylamine is added in portions at 50° C. to 30 g (117.1 mmol) of EDTA-bisanhydride, suspended in 200 ml of dimethylformamide and 50 ml of pyridine, and it is stirred for 6 hours at 50° C. 10 ml of water is added, it is stirred for 10 minutes at 50° C., and the residue is evaporated to the dry state. The residue is taken up in a little water and brought to pH 4 with glacial acetic acid. The insoluble precipitate is filtered off and chromatographed on RP-18 (mobile solvent: acetonitrile/water/gradient).

Yield: 9.58 g (61% of theory) of a colorless solid

Water content: 8%

Elementary analysis:

Cld: C, 33.64; H, 3.59; N, 5.35; F, 41.12.

Fnd: C, 33.51; H, 3.69; N, 5.44; F, 41.24.

### b) Ethylenediamine-N,N-tetraacetic acid-N-(1H,1H,2H,2H,4H,4H,5H,5H-3-oxa-perfluorotridecyl)-monoamide, calcium salt, sodium salt

9.0 g (11.46 mmol) of the title substance of Example 19a is suspended in 300 ml of water, and 11.4 ml of 1N aqueous sodium hydroxide solution is added. Then, 1.15 g (11.46 mmol) of calcium carbonate is added and stirred for 5 hours at 50° C. The solution is filtered, and the filtrate is freeze-dried.

Yield: 9.7 g (100% of theory) of a colorless, amorphous solid

Water content: 7.5%

Elementary analysis:

Cld: C, 31.25; H, 2.98; N, 4.97; F, 38.20; Na, 2.72; Ca, 4.74.

Fnd: C, 31.40; H, 3.09; N, 5.10; F, 38.07; Na, 2.81; Ca, 4.82.

### c) Production of a formulation that consists of gadolinium complex I and ethylenediamine-N,N-tetraacetic acid-N-(1H,1H,2H,2H,4H,4H,5H,5H-3-oxa-pefluorotridecyl)-monoamide, calcium salt, sodium salt

2.54 g (3.01 mmol) of the title compound of Example 19b is added to 43 ml of a solution of gadolinium complex I (280 mmol/L), dissolved in 0.45% of aqueous common salt solution (pH 7.4; 0.25 mg/L of CaNa₃DTPA), and, with a 0.9% aqueous common salt solution, it yields a total of 121 ml. It is heated for 2 hours at 60° C. in an ultrasound bath. The solution is cooled to room temperature and set at pH 7.4 with aqueous 2N sodium hydroxide solution. It is filtered through a 0.2 μm filter, and the filtrate is decanted into vials. A solution that is thus produced can be used directly for biological experiments. (The concentration is 100 mmol of Gd/L.)

### EXAMPLE 20

### a) 1H,1H,2H,2H-Perfluorodecyl-(2,2-dimethyl-5-hydroxy-1,3-dioxepan-6-yl)-ether

30 g (64.64 mmol) of 1H,1H,2H,2H-perfluorodecanol is dissolved in 200 ml of tetrahydrofuran, and 1.68 g (70 mmol) of sodium hydride is added at 0° C. It is stirred for 2 hours at room temperature, then for 4 hours at 60° C. The solution is poured into a metal autoclave, then 9.31 g (64.64 mmol) of 2,2-dimethyl-1,3,6-trioxabicyclo[5.1.0]octane is added and then heated to 150° C. for 10 hours. The reaction solution is poured onto ice water and extracted twice with diethyl ether. The combined organic phases are evaporated to the dry state, and the residue is chromatographed on silica gel (mobile solvent: dichloromethane/acetone=10:1).

Yield: 16.12 g (41% of theory) of a colorless solid

Elementary analysis:

Cld: C, 33.57; H, 2.82; F, 53.10.

Fnd: C, 33.69; H, 2.90; F, 53.35.

### b) 1H,1H,2H,2H-Perfluorodecyl-(1-hydroxymethyl-2,3-dihydroxypropyl)-ether

15 g (24.66 mmol) of the title compound of Example 20a is dissolved in 300 ml of ethanol, and 30 ml of 10% aqueous hydrochloric acid is added. It is heated for 5 hours under reflux. It is set at pH 7 with sodium hydroxide solution, then evaporated to the dry state, and the residue is chromatographed on RP-18 (mobile solvent: acetonitrile/water/gradient).

Yield: 12.75 g (91% of theory) of a colorless solid

Water content: 4.5%

Elementary analysis:

Cld: C, 29.59; H, 2.31; F, 56.84.

Fnd: C, 29.48; H, 2.37; F, 56.99.

### c) Production of a formulation that consists of gadolinium complex IV and 1H,1H,2H,2H-perfluorodecyl-(1-hydroxymethyl-2,3-dihydroxypropyl)-ether

9.46 g (16.65 mmol) of the title compound of Example 20b is added to 37 ml of a solution of gadolinium complex IV (300 mmol/L), dissolved in 0.45% of aqueous common salt solution (pH 7.4; 0.25 mg/L of CaNa₃DTPA), and, with a 0.9% aqueous common salt solution, it yields a total of 111 ml. It is heated for 2 hours at 60° C. in an ultrasound bath. The solution is cooled to room temperature and set at pH 7.4 with aqueous 2N sodium hydroxide solution. It is filtered through a 0.2 μm filter, and the filtrate is decanted into vials. A solution that is thus produced can be used directly for biological experiments. (The concentration is 100 mmol of Gd/L.)

### EXAMPLE 21

### a) 1H,1H,2H,2H-Perfluorodecyl-[1,2-bis(2,2-dimethyl-1,3-dioxolan-4-yl)-2-hydroxyethyl]-ether

30 g (64.64 mmol) of 1H,1H,2H,2H-perfluorodecanol is dissolved in 200 ml of tetrahydrofuran, and 1.68 g (70 mmol) of sodium hydride is added at 0° C. It is stirred for 2 hours at room temperature, then for 4 hours at 60° C. The solution is poured into a metal autoclave, then 15.78 g (64.64 mmol) of 1,2-bis-(2,2-dimethyl-1,3-dioxolan-4-yl)-oxiran is added, and then it is heated for 10 hours to 150° C. The reaction solution is poured onto ice water and extracted twice with diethyl ether. The combined organic phases are evaporated to the dry state, and the residue is chromatographed on silica gel (mobile solvent: dichloromethane/acetone=10:1).

Yield: 14.2 g (31% of theory) of a colorless solid

Elementary analysis:

Cld: C, 37.30; H, 3.56; F, 45.59.

Fnd: C, 37.48; H, 3.66; F, 45.71.

### b) 1H,1H,2H,2H-Perfluorodecyl-[1,2-bis(1,2-dihydroxy-ethyl)-2-hydroxyethyl]-ether

14 g (19.76 mmol) of the title compound of Example 21a is dissolved in 300 ml of ethanol, and 30 ml of 10% aqueous hydrochloric acid is added. It is heated under reflux for 5 hours. It is set at pH 7 with sodium hydroxide solution, then evaporated to the dry state, and the residue is chromatographed on RP-18 (mobile solvent: acetonitrile/water/gradient).

Yield: 10.55 g (85% of theory) of a colorless solid

Water-content: 3.2%

Elementary analysis:

Cld: C, 30.59; H, 2.73; F, 51.41.

Fnd: C, 30.73; H, 2.81; F, 51.58.

### c) Production of a formulation that consists of gadolinium complex II and 1H,1H,2H,2H-perfluorodecyl-[1,2-bis(1,2-dihydroxy-ethyl)-2-hydroxyethyl]-ether

11.98 g (19.07 mmol) of the title compound of Example 21b is added to 41 ml of a solution of gadolinium complex II (300 mmol/L), dissolved in 0.45% aqueous common salt solution (pH 7.4; 0.25 mg/L of CaNa₃DTPA), and, with a 0. 9% aqueous common salt solution, it yields a total of 64 ml. It is heated for 2 hours at 60° C. in an ultrasound bath. The solution is cooled to room temperature and set at pH 7.4 with aqueous 2N sodium hydroxide solution. It is filtered through a 0.2 βm filter, and the filtrate is decanted into vials. A solution that is thus produced can be used directly for biological experiments. (The concentration is 200 mmol of Gd/L.)

### EXAMPLE 22

### a) Perfluorooctylsulfonic acid-N,N-bis[(8-sulfuric acid-monoester, sodium salt)-3,6-dioxaoctyl]-amide

13.54 g (17.73 mmol) of the title compound of Example 15a is dissolved in 300 ml of chloroform, and 2.82 g (17.73 mmol) of sulfur trioxide-pyridine complex is added at 0° C. It is stirred for one hour at 0° C., and then it is evaporated to the dry state in a vacuum. The residue is dissolved in 300 ml of ethanol and mixed with 17.8 ml of 1N aqueous sodium hydroxide solution. The solution is evaporated to the dry state, and the residue is-dried in a vacuum (60° C./2 hours)

Yield: 17.15 g (quantitative)

Elementary analysis:

Cld: C, 24.83; H, 2.50; F, 33.83; N, 1.45; S, 9.94; Na, 4.75.

Fnd: C, 24.96; H, 2.62; F, 33.97; N, 1.53; S, 10.05; Na, 4.86.

### b) Production of a formulation that consists of gadolinium complex I and perfluorooctylsulfonic acid-N,N-bis[(8-sulfuric acid-monoester, sodium salt)-3,6-dioxaoctyl]-amide

142.29 g (147.06 mmol) of the title compound of Example 22a is added to 43 ml of a solution of gadolinium complex I (380 mmol/L), dissolved in 0.45% aqueous common salt solution (pH 7.4; 0.25 mg/L of CaNa₃DTPA), and, with a 0.9% aqueous common salt solution, it yields a total of 164 ml. It is heated for 2 hours at 60° C. in an ultrasound bath. The solution is cooled to room temperature and set at pH 7.4 with aqueous 2N sodium hydroxide solution. It is filtered through a 0.2 μm filter, and the filtrate is decanted into vials. A solution that is thus produced can be used directly for biological experiments. (The concentration is 100 mmol of Gd/L.)

### EXAMPLE 23

### a) 2-(2H,2H,3H,3H,5,5H,6H,6H-1,4-Dioxaperfluorotetradec-1-yl)-succinic acid diethyl ester

30 g (59.03 mmol) of 1H,1H,2H,2H,4H,4H,5H,5H-3-oxa-perfluorotridecanol is added in 300 ml of tetrahydrofuran, and 1.68 g (70 mmol) of sodium hydride is added at 0° C. It is stirred for one hour at 0° C., then for 5 hours at 40° C. 20.25 g (80 mmol) of 2-bromo-succinic acid diethyl ester is added in drops within 10 minutes to this 40° C. solution, and then it is stirred for 12 hours at this temperature. 500 ml of ice water is added, and it is extracted twice with 300 ml of diethyl ether. The combined organic phases are evaporated to the dry state in a vacuum, and the residue is chromatographed on silica gel (mobile solvent: n-hexane/ethanol=20:1).

Yield: 12.05 g (30% of theory)

Elementary analysis:

Cld: C, 35.31; H, 3.11; F, 47.47.

Fnd: C, 35.19; H, 3.20; F, 47.59.

### b) 2-(2H,2H,3H,3H,5H,5H,6H,6H-1,4-dioxa-perfluorotetradec-1-yl)-succinic acid, disodium salt

50 ml of 3N aqueous sodium hydroxide solution is added to 11.5 g (16.90 mmol) of the title compound, dissolved in 300 ml of methanol, and it is refluxed for 8 hours. It is evaporated to the dry state, and the residue is taken up in 300 ml of water. The aqueous phase is extracted twice with 300 ml of diethyl ether. The aqueous phase is acidified to pH 1 with concentrated hydrochloric acid, and it is extracted twice with 300 ml of chloroform. The combined chloroform phases are dried on magnesium sulfate and evaporated to the dry state. The residue is dissolved in 300 ml of water and set at pH 7.4 with 5% aqueous sodium hydroxide solution. Then, it is freeze-dried.

Yield: 10.50 g (93% of theory) of a colorless, amorphous solid

Water content: 5.7%

Elementary analysis:

Cld: C, 28.76; H, 1.66; F, 48.33; Na, 6.88.

Fnd: C, 28.88; H, 1.71; F, 48.25; Na, 6.95.

### c) Production of a formulation that consists of gadolinium complex II and 2-(2H,2H,3H,3H,5H,5H,6H,6H-1,4-dioxa-perfluorotetradec-1-yl)-succinic acid, disodium salt

1.14 g (1.71 mmol) of the title compound of Example 23b is added to 57 ml of a solution of gadolinium complex II (300 mmol/L), dissolved in 0.45% aqueous common salt solution (pH 7.4; 0.25 mg/L of CaNa₃DTPA), and, with a 0.9% aqueous common salt solution, it yields a total of 154 ml. It is heated for 2 hours at 60° C. in an ultrasound bath. The solution is cooled to room temperature and set at pH 7.4 with aqueous 2N sodium hydroxide solution. It is filtered through a 0.2 μm filter, and the filtrate is decanted into vials. A solution that is thus produced can be used directly for biological experiments. (The concentration is 100 mmol of Gd/L.)

### EXAMPLE 24

### a) 2H,2H,4H,4H,5H,5H-3-Oxa-perfluorotridecanoic acid-N-(succin-2-yl)-amide

16.51 g (80 mmol) of N,N′-dicyclohexylcarbodiimide is added at 0° C. to 20 g (38.30 mmol) of 2H,2H,4H,4H,5H,5H-3-oxa-perfluorotridecanoic acid and 9.21 g (80 mmol) of N-hydroxysuccinimide, dissolved in 150 ml of dimethylformamide, and it is stirred for 3 hours at this temperature. A solution of 5.10 g (38.30 mmol) of L-aspartic acid, dissolved in 300 ml of 5% aqueous sodium carbonate solution and cooled to 0° C., is added to the active ester solution that is thus produced, and it is stirred for 2 hours at 0° C. It is poured onto 500 ml of ice water, precipitated dicyclohexylurea is filtered out, and then set at pH 1 with concentrated hydrochloric acid. It is extracted three times with 300 ml of chloroform. The combined, organic phases are evaporated to the dry state, and the residue is chromatographed on RP-18 (mobile solvent: acetonitrile/water/gradient). The dioic acid that is thus obtained is dissolved in 400 ml of water and set at pH 7.4 with 1N aqueous sodium hydroxide solution. It is filtered, and the filtrate is freeze-dried.

Water content: 6.3%

Yield: 21.13 g (81% of theory) of a colorless amorphous powder

Elementary analysis:

Cld: C, 28.21; H, 1.48; N, 2.06; F, 47.41; Na, 6.75.

Fnd: C, 28.30; H, 1.53; N, 2.11; F, 47.53; Na, 6.83.

### b) Production of a formulation that consists of gadolinium complex III and 2H,2H,4H,4H,5H,5H-3-oxa-perfluorotridecanoic acid-N-(succin-2-yl)-amide

422 mg (0.62 mmol) of the title compound of Example 24a is added to 37 ml of a solution of gadolinium complex III (300 mmol/L), dissolved in 0.45% of aqueous common salt solution (pH 7.4; 0.25 mg/L of CaNa₃DTPA), and, with a 0.9% aqueous common salt solution, it yields a total of 111 ml. It is heated for 2 hours at 60° C. in an ultrasound bath. The solution is cooled to room temperature and set at pH 7.4 with aqueous 2N sodium hydroxide solution. It is filtered through a 0.2 μm filter, and the filtrate is decanted into vials. A solution that is thus produced can be used directly for biological experiments. (The concentration is 100 mmol of Gd/L.)

### EXAMPLE 25

### Production of a formulation that consists of gadolinium complex VII and perfluorooctanesulfonic acid, sodium salt

A solution of 1.34 g (2.69 mmol) of perfluorooctanesulfonic acid, sodium salt, dissolved in 200 ml of ethanol, is added to 43 ml of a solution of gadolinium complex VII (250 mmol/L), dissolved in 0.45% sodium chloride solution (pH 7.4/0.25 mg/L of CaNa₃DTPA), and it is stirred for 2 hours at 50° C. The solution is evaporated to the dry state in a vacuum, and, with distilled water, the residue yields a total of 108 ml. It is stirred for 10 minutes at 40° C., and filtered through a 0.2 μm filter. The filtrate is decanted into vials. A solution that is thus produced can be used directly for biological experiments. (The concentration is 100 mmol of Gd/L.)

### EXAMPLE 26

### Production of a formulation that consists of gadolinium complex VIII and perfluorodecanesulfonic acid, sodium salt

3.03 g (5.06 mmol) of perfluorodecanesulfonic acid, sodium salt, is added to 49 ml of a solution of gadolinium complex VIII (310 mmol/L), dissolved in 0.45% of aqueous sodium chloride solution), and it is heated for 10 minutes in the microwave. The solution is cooled to room temperature, filtered though a 0.2 μm filter, and the filtrate is decanted into vials. A solution that is thus produced can be used directly for biological experiments. (The concentration is 310 mmol of Gd/L.)

### EXAMPLE 27

### a) (1H,1H,2H,2H-Perfluorodecyl)-5-[(1,3-dicarboxy, disodium salt)-phenyl]-ether

42.5 g (80.62 mmol) of the title compound of Example 14a is added to 20 g (80.62 mmol) of the trisodium salt of 50-hydroxy-isophthalic acid in 300 ml of dimethylformamide, and it is stirred for 10 hours at 60° C. It is poured onto ice water and set at pH 1 with concentrated hydrochloric acid. It is extracted 3 times with 300 ml of chloroform. The combined, organic phases are concentrated by evaporation, and the residue is chromatographed on RP-18 (mobile solvent: acetonitrile/water/gradient). The dioic acid that is thus purified is dissolved in 400 ml of water, and the pH is brought to pH 7.4 with 1N aqueous sodium hydroxide solution. It is filtered, and the filtrate is freeze-dried.

Yield: 20.05 g (37% of theory) of a colorless, amorphous solid

Water content: 5.0%

Elementary analysis:

Cld: C, 32.16; H, 1.05; F, 48.05; Na, 6.84.

Fnd: C, 32.30; H, 1.15; F, 48.20; Na, 6.95.

### b) Production of a formulation that consists of gadolinium complex IV and (1H,1H,2H,2H-perflurododecyl)-5-[(1,3-dicarboxy, disodium salt)-phenyl]-ether

6.86 g (10.2 mmol) of the title compound of Example 27a is added to 51 ml of a solution of gadolinium complex IV (300 mmol/L), dissolved in 0.45% aqueous common salt solution (pH=74; 0.25 mg/L of CaNa₃DTPA), and, with a 0.9% aqueous common salt solution, it yields a total of 153 ml. It is heated for 2 hours at 60° C. in an ultrasound bath. The solution is cooled to room temperature and set at pH 7.4 with aqueous 2N sodium hydroxide solution. It is filtered by a 0.2 μm filter, and the filtrate is decanted into vials. A solution that is thus produced can be used directly for biological experiments. (The concentration is 100 mmol of Gd/L.)

### EXAMPLE 28

### Production of a formulation that consists of gadolinium complex III and 3-oxa-2H,2H,4H,4H,5H,5H-perfluorotridecanoic acid, sodium salt

434 mg (0.55 mmol) of the title compound of Example 11a is added to 4 ml of a solution of gadolinium complex III (320 mmol/L), dissolved in 0.45% of aqueous common salt solution (pH 7.4; 0.25 mg/L of CaNa₃DTPA), and, with a 0.9% aqueous common salt solution, it yields a total of 12.8 ml. It is heated for 2 hours at 60° C. in an ultrasound bath. The solution is cooled to room temperature and set at pH 7.4 with aqueous 2N sodium hydroxide solution. It is filtered through a 0.2 μm filter, and the filtrate is decanted into vials. A solution that is thus produced can be used directly for biological experiments. (The concentration is 100 mmol of Gd/L.)

### EXAMPLE 29

### a) (Adamant-1-yl)-3-oxa-propionic acid-1-butyl ester

29.26 g (150 mmol) of bromoacetic acid-tert-butyl ester is added at 0° C. to 15.22 g (100 mmol) of 1-adamantanol in 300 ml of 50% aqueous potassium hydroxide solution, 200 ml of toluene, and it is stirred vigorously for 2 hours. It is poured onto 1500 ml of water and extracted twice with 300 ml of diethyl ether. The combined organic phases are dried on magnesium sulfate and evaporated to the dry state in a vacuum. The residue is chromatographed on silica gel (mobile solvent: n-hexane-diethyl ether 20:1).

Yield: 21.58 g (81% of theory) of a viscous, colorless oil

Elementary analysis:

Cld: C, 72.14; H, 9.84.

Fnd: C, 72.26; H, 9.95.

### b) (Adamant-1-yl)-3-oxa-propionic acid

20 g (75 mmol) of the title compound of Example 29a is dissolved at 0C in 200 ml of trifluoroacetic acid and stirred for 8 hours at room temperature. It is evaporated to the dry state, and the residue is crystallized from diisopropyl ether.

Yield: 14.68 g (93% of theory) of colorless flakes

Elementary analysis:

Cld: C, 68.55; H, 8.63.

Fnd: C, 68.41; H, 8.74.

### c) 1-(Perfluorooctylsulfonyl)-4-[(adamant-1-yl)-oxapropionyl]-piperazine

14 g (66.6 mmol) of the title compound of Example 29b and 37.50 g (66.6 mmol) of 1-perfluorooctylsulfonyl-piperazine are dissolved in 300 ml of tetrahydrofuran, and 32.15 g (130 mmol) of 1,2-dihydro-2-ethoxyquinoline-1-carboxylic acid ethyl ester (=EEDQ) is added at 0° C. It is stirred for 5 hours at room temperature. The solution is evaporated to the dry state in a vacuum, and the residue is chromatographed on silica gel (mobile solvent: dichloromethane/diethyl ether 30:1).

Yield: 43.05 g (85% of theory) of a colorless solid

Elementary analysis:

Cld: C, 37.90; H, 3.31; N, 3.68; S, 4.22; F, 42.47.

Fnd: C, 38.04; H, 3.42; N, 3.49; S, 4.11; F, 42.30.

### d) Preparation that consists of 0.5 part of gadolinium complex I and 0.5 part of an inclusion compound that consists of β-cyclodextrin-hydrate and 1-(perfluoroctylsulfonyl)-4-[(adamant-1-yl)-oxapropionyl]-piperazine

6.81 g (8.96 mmol) of the title compound of Example 29c, and 10.33 g (8.96) of β-cyclodextrin monohydrate are added to 32 ml of a solution of gadolinium complex 1 (280 mmol/L), dissolved in 0.45% aqueous common salt solution (pH 7.4; 0.25 mg/L of CaNa₃DTPA), and, with a 0.9% aqueous common salt solution, it yields a total of 98 ml. It is heated for 2 hours at 60° C. in an ultrasound bath. The solution is cooled to room temperature and set at pH 7.4 with aqueous 2N sodium hydroxide solution. It is filtered by a 0.2 μm filter, and the filtrate is decanted into vials. A solution that is thus produced can be used directly for biological experiments. (The Gd concentration is 100 mmol of Gd/L.)

### EXAMPLE 30

### a) 3-Oxa-2H,2H,4H,4H,5H,5H-perfluorotridecanoic acid-N-(1-adamantyl)-amide

30.95 g (150 mmol) of N,N-dicyclohexyl carbodiimide is added at 0° C. to 15.12 g (100 mmol) of 1-amino-adamantan, 52.21 (100 mmol) of 3-oxa-2H,2H,4H,4H,4H,5H,5H-perfluorotridecanoic acid and 11.5 g (100 mmol) of N-hydroxysuccinimide, dissolved in 300 ml of tetrahydrofuran. It is stirred for 2 hours at 0° C., then for 6 hours at room temperature. The precipitated urea is filtered out, the filtrate is evaporated to the dry state, and the residue is chromatographed on silica gel (mobile solvent: dichloromethane/acetone=30:1).

Yield: 54.4 g (83% of theory) of a waxy solid

Elementary analysis:

Cld: C, 40.32; H, 3.38; N, 2.14; F, 49.28.

Fnd: C, 40.47; H, 3.49; N, 2.03; F, 49.09.

### b) Preparation of 0.6 part of gadolinium complex II and 0.4 part of an inclusion compound that consists of β-cyclodextrin-hydrate and 3-oxa-2H,2H,4H,4H,5H,5H-perfluorodotridecanoic acid-N-(1-adamantyl)-amide

4.48 g (6.83 mmol) of the title compound of Example 30a and 7.87 g (6.83 mmol) of β-cyclodextrin monohydrate are added to 41 ml of a solution of the gadolinium complex II (250 mmol/L), dissolved in 0.45% aqueous common salt solution (pH 7.4; 0.25 mg/L of CaNa₃DTPA), and, with a 0.9% aqueous common salt solution, it yields a total of 103 ml. It is heated for 2 hours at 60° C. in an ultrasound bath. The solution is cooled to room temperature and set at pH 7.4 with aqueous 2N sodium hydroxide solution. It is filtered through a 0.2 μm filter, and the filtrate is decanted into vials. A solution that is thus produced can be used directly for biological experiments. (The Gd-concentration is 100 mmol of Gd/L.)

### EXAMPLE 31

### a) 2-[N-(Ethyl)-N-(perfluorooctylsulfony)-amino]-acetic acid-N-(adamantyl)-amide

30.95 g (150 mmol) of N,N-dicyclohexylcarbodiimide is added at 0° C. to 15.12 g (100 mmol) of 1-aminoadamantan, 58.52 g (100 mmol) of N-(ethyl)-N-(prefluorooctylsulfonyl)-aminoacetic acid and 11.51 g (100 mmol) of N-hydroxysuccinimide, dissolved in 300 ml of tetrahydrofuran. It is stirred for 2 hours at 0° C., then for 6 hours at room temperature. Precipitated urea is filtered out, the filtrate is evaporated to the dry state, and the residue is chromatographed on silica gel (mobile solvent: dichloromethane/acetone=30:1).

Yield: 55.65 g (79% of theory) of an amorphous solid

Elementary analysis:

Cld: C, 37.51; H, 3.29; F, 45.85; N, 1.99; S, 4.55.

Fnd: C, 37.64; H, 3.41; F, 45.99; N, 2.13; S, 4.43.

### b) Preparation that consists of 0.6 part of gadolinium complex I and 0.4 part of the inclusion compound that consists of β-cyclodextrin-hydrate and 2-[N-(ethyl)-N-(perfluorooctyl-sulfonyl)-amino]-acetic acid-N-(1-adamantyl)-amide

4.20 g (5.97 mmol) of the title compound of Example 31a and 6.88 g (5.97 mmol) of β-cyclodextrin monohydrate are added to 32 ml of a solution of gadolinium complex I (280 mmol/L), dissolved in 0.45% aqueous common salt solution (pH 7.4; 0.25 mg/L of CaNa₃DTPA), and, with a 0.9% aqueous common salt solution, it yields a total of 90 ml. It is heated for 2 hours at 60° C. in an ultrasound bath. The solution is cooled to room temperature and set at pH 7.4 with aqueous 2N sodium hydroxide solution. It is filtered through a 0.2 μm filter, and the filtrate is decanted into vials. A solution that is thus produced can be used directly for biological experiments. (The Gd concentration is 100 mmol of Gd/L.)

### EXAMPLE 32

### Lymph Node Concentration in Guinea Pigs After Interstitial Administration

A galenical formulation that consists of complex I (Gd-GlyMe-DOTA-perfluorooctylsulfonamide) and the compound of Example 11 (mannose-perfluorooctylsulfonamide; proportion 40% mol) was examined 30 and 60 minutes as well as 24 hours after subcutaneous administration (10 μmol of total gadolinium/kg of KGW (body weight), hind paw, s.c.) in guinea pigs with stimulated lymph nodes (complete Freund adjuvant; in each case 0.1 ml i.m. in the right and left upper and lower leg; 2 weeks before the test substance is administered) with respect to their lymph node concentration in three successive lymph node stations (popliteal, inguinal, iliac). In this case, the results that are listed below were obtained: (determination of the gadolinium concentration by means of ICP-AES, MW±SD, n-3):

| | Gd Content in | | | | | |
|---|---|---|---|---|---|---|
| | [μmol/l] | | | [% dose/g of tissue] | | |
| | | | 24 | | | 24 |
| | 30′ | 60′ | hours | 30′ | 60′ | hours |
| | p.i. | p.i. | p.i. | p.i. | p.i. | p.i. |
| popliteal | 1643 ± 450 | 1114 ± 470 | 131 ± 31 | 48.1 ± 13.2 | 32.6 ± 13.8 | 3.8 ± 0.9 |
| ing. | 784 ± 302 | 779 ± 414 | 144 ± 72 | 23.1 ± 8.8 | 22.8 ± 12.1 | 4.2 ± 2.1 |
| prof. | | | | | | |
| iliac | 685 ± 262 | 575 ± 349 | 120 ± 8 | 20.1 ± 7.7 | 16.8 ± 10.2 | 3.5 ± 0.2 |
| blood | 4 ± 2 | 6 ± 1 | 2 ± 0 | 0.1 ± 0.1 | 0.2 ± 0.0 | 0.1 ± 0.0 |
| urine | 0 ± 0 | 1 ± 1 | 1 ± 1 | 0.0 ± 0.0 | 0.0 ± 0.0 | 0.0 ± 0.0 |

### EXAMPLE 33

### Retention of the Opacifying Metal at the Injection Site After Interstitial Administration in Guinea Pigs

After s.c. administration of 10 μmol of total gadolinium/kg of KGW of a galenical formulation that consists of complex I (Gd-GlyMe-DOTA-perfluorooctylsulfonamide) and the compound of Example 11 (mannose-perfluorooctylsulfonamide; proportion 40 mol %) in the guinea pig paw, the retention of the metal at the injection site was examined at various times (MW=SD, n=3).

Gd Content at the
Injection Site (Paw)

| | [μmol/l] | [% dose] |
|---|---|---|
| 30 minutes p.i. | 1607 ± 184 | 101.6 ± 19.3 |
| 90 minutes p.i. | 1219 ± 173 | 79.9 ± 13.2 |
| 24 hours p.i. | 57 ± 7 | 3.5 ± 0.5 |
| 7 days p.i. | 49 ± 22 | 3.1 ± 1.5 |

### EXAMPLE 34

### Organ Distribution of the Opacifying Metal After Interstitial Administration in Guinea Pigs

After subcutaneous administration of 10 μmol of total gadolinium/kg of KGW of a galenical formulation that consists of complex I (Gd-GlyMe-DOTA-perfluorooctylsulfonamide) and the compound of Example 11 (mannose-perfluorooctylsulfonamide; proportion of 40 mol %) in the hind foot of the guinea pigs with stimulated lymph nodes (complete Freund adjuvant; in each case 0.1 ml i.m. in the right and left upper and lower leg; 2 weeks before the test substance is administered), the retention of the metal in the liver as well as in the kidneys and spleen was examined 7 days after administration (MW±SD, n=3).

| 7 days p.i. | Gd Content in [% dose] |
|---|---|
| Liver | 2.4 ± 0.6 |
| Kidneys | 0.2 ± 0.0 |
| Spleen | 0.0 ± 0.0 |

### EXAMPLE 35

### Lymph Node Visualization (MRT) After Intravenous Administration of the Contrast Medium in Guinea Pigs

FIG. 1 shows MR images of popliteal, inguinal and iliac lymph nodes both before (baseline: precontrast) and 60 minutes or 24 hours after intravenous administration of 100 μmol of Gd/kg of KGW of a galenical formulation that consists of Gd-GlyMe-DOTA-perfluorooctyl-sulfonamide (complex I) and mannose-perfluorooctylsulfonamide (Example 11; proportion of 40 mol %) in guinea pigs with stimulated lymph nodes (complete Freund adjuvant; in each case 0.1 ml i.m. in the right and left upper and lower leg; 2 weeks before the test substance is administered). The T₁-weighted, gradient echo images (TR 10 ms, TE 5 ms, 40°) illustrate the strong signal increase in the various lymph nodes (arrows) in comparison to the precontrast image. In the table, the corresponding enhancement values (in % in the precontrast value) and signal intensity quotients of lymph nodes/muscle are presented (MW±SD n=3).

| | | Complex I + Example 11; 100 μmol of Gd/kg (n = 3) | | | | | |
|---|---|---|---|---|---|---|---|
| Time p.i. | | Enhancement [%] | | | Sl Lymph Nodes/Muscle | | |
| [min] | | popliteal | ing. profund | iliac | popliteal | ing. profund | iliac |
| 0 | min | — | — | — | 0.9 ± 0.2 | 1.2 ± 0.1 | 1.0 ± 0.1 |
| 60 | min | 170 ± 38 | 123 ± 20 | 122 ± 13 | 1.2 ± 0.2 | 1.4 ± 0.1 | 1.2 ± 0.1 |
| 24 | h | 114 ± 37 | 69 ± 7 | 67 ± 14 | 1.3 ± 0.4 | 1.4 ± 0.0 | 1.2 ± 0.0 |

### EXAMPLE 36

### Lymph Node Visualization (MRT) After Intravenous Administration of the Contrast Medium in Guinea Pigs

FIG. 2 shows MR images of popliteal, inguinal and iliac lymph nodes both before (baseline: precontrast) and 5, 60, 90 minutes or 24 hours after intravenous administration of 100 μmol of Gd/kg of KGW of a galenical formulation that consists of complex I (Gd-GlyMe-DOTA-perfluorooctyl-sulfonamide) and 3-oxa-2H2H4H4H5H5H-perfluorotridecanoic acid (proportion 10, 20 or 40 mol %) in guinea pigs with stimulated lymph nodes (complete Freund adjuvant; in each case 0.1 ml i.m. in the right and left upper and lower leg; 2 weeks before the test substance is administered). The T₁-weighted, gradient echo images (TR 10 ms, TE 5 ms, α=40°) illustrate the strong signal increase in the various lymph nodes (arrows) in comparison to the precontrast image. In Table 1, the corresponding enhancement values (in % in the precontrast value) and signal intensity quotients of lymph nodes/muscles are presented (MW±SD, n=3).

### EXAMPLE 37

### Lymph Node Visualization (MRT) After Intravenous Administration of the Contrast Medium in Guinea Pigs

FIG. 3 shows MR images of popliteal, inguinal and iliac lymph nodes both before (baseline: precontrast) and 60 minutes or 18 hours after intravenous administration of 200 μmol of Gd/kg of KGW of a galenical formulation that consists of complex III (Gd-GlyMe-DOTA-trimer-perfluorooctyl-oxadecylamide) and the compound of Example 11 (mannose-perfluorooctylsulfonamide; proportion: 40 mol %) in guinea pigs with stimulated lymph nodes (complete Freund adjuvant; in each case 0.1 ml i.m. in the right and left upper and lower leg; 2 weeks before the test substance is administered). The T₁-weighted, gradient echo images (TR 10 ms, TE 5 ms, α 40°) illustrate the strong signal increase in the various lymph nodes (arrows) in comparison to the precontrast image. In the Table, the corresponding enhancement values (in % in precontrast value) as well as signal intensity quotients of lymph nodes/muscles are presented (MW±SD, n=3).

| | | | | Si Lymph | |
|---|---|---|---|---|---|
| | | Enhancement [%] | | Nodes/Muscles | |
| Time p.i. | | popliteal | iliac | popliteal | iliac |
| 0 | min | — | — | 0.7 ± 2 | 1.1 ± 0.1 |
| 60 | min | 263 ± 167 | 139 ± 43 | 1.0 ± 0.1 | 1.1 ± 0.1 |
| 18 | h | 21 ± 21 | 18 ± 10 | 0.8 ± 0.2 | 1.2 ± 0.2 |

### EXAMPLE 38

### Organ Distribution (Including Lymph Node Concentration) After Intravenous Administration of the Contrast Medium in Prostate-Cancer-Carrying Rats

After intravenous administration of 180 μmol of total gadolinium/kg of KGW of a galenical formulation that consists of Gd-GlyMe-DOTA-perfluorooctylsulfonamide (complex I) and mannose-perfluorooctylsulfonamide (Example 11; proportion 40 mol %) in rats (Cop-inbreeding Dunning R3327 MAT-Lu prostate cancer i.m.—implanted 12 days earlier), the metal content in various organs as well as in the lymph nodes (pooled as mesenteric and peripheral lymph nodes) was determined 10 minutes, 1 and 24 hours after administration (MW±SD, n=3).

| | Complex I + Substance of Example 11 | | | | | |
|---|---|---|---|---|---|---|
| | Gd Concentration | | | | | |
| | [μmol/l] | | | % Dose | | |
| | 10 min | 1 h | 24 h | 10 min | 1 h | 24 h |
| | p.i. | p.i. | p.i. | p.i. | p.i. | p.i. |
| Liver | 767 ± 37 | 683 ± 27 | 1350 ± 23 | 14.12 ± 1.74 | 11.49 ± 0.33 | 22.25 ± 0.84 |
| Spleen | 713 ± 156 | 862 ± 17 | 1140 ± 60 | 0.65 ± 0.16 | 0.87 ± 0.03 | 1.22 ± 0.10 |
| Pancreas | 527 ± 118 | 407 ± 1 | 213 ± 16 | 1.03 ± 0.10 | 0.65 ± 0.03 | 0.39 ± 0.07 |
| Kidney | 1116 ± 47 | 875 ± 68 | 1059 ± 48 | 3.42 ± 0.19 | 2.81 ± 0.13 | 3.98 ± 0.28 |
| Lung | 1309 ± 125 | 980 ± 21 | 614 ± 22 | 3.93 ± 0.65 | 2.45 ± 0.02 | 1.34 ± 0.05 |
| Heart | 604 ± 46 | 407 ± 36 | 162 ± 7 | 0.96 ± 0.17 | 0.57 ± 0.08 | 0.20 ± 0.02 |
| Brain | 64 ± 5 | 39 ± 2 | 24 ± 2 | 0.20 ± 0.01 | 0.12 ± 0.01 | 0.08 ± 0.01 |
| Muscle**** | 98 ± 18 | 109 ± 8 | 44 ± 3 | 0.09 ± 0.02 | 0.10 ± 0.00 | 0.04 ± 0.01 |
| Tumor | 97 ± 5 | 196 ± 7 | 248 ± 4 | 0.10 ± 0.04 | 0.50 ± 0.20 | 0.39 ± 0.08 |
| Femur | 176 ± 22 | 162 ± 16 | 120 ± 6 | 0.74 ± 0.06 | 0.69 ± 0.00 | 0.53 ± 0.05 |
| Mes. LK | 406 ± 44 | 663 ± 71 | 574 ± 55 | 0.12 ± 0.03 | 0.17 ± 0.00 | 0.21 ± 0.02 |
| Periph. LK | 205 ± 58 | 465 ± 33 | 400 ± 34 | 0.10 ± 0.03 | 0.26 ± 0.02 | 0.23 ± 0.04 |
| Stomach | 331 ± 18 | 325 ± 10 | 255 ± 16 | 0.92 ± 0.07 | 1.01 ± 0.02 | 0.70 ± 0.03 |
| (emptied) | | | | | | |
| Intestine | 441 ± 42 | 538 ± 26 | 4.34 ± 14 | 4.15 ± 0.63 | 4.39 ± 0.16 | 3.56 ± 0.04 |
| (emptied) | | | | | | |
| (Blood)* | 914 ± 158 | 546 ± — | 234 ± 11 | 29.88 ± 5.09 | 17.88 ± — | 7.66 ± 0.34 |
| Remainder of | 346 ± 14 | 397 ± 38 | 245 ± 2 | 67.65 ± 2.70 | 77.18 ± 4.67 | 46.14 ± 0.58 |
| the body | | | | | | |
| Urine 0-24 h | — | — | 11 ± 2 | — | — | 1.68 ± 0.34 |
| Feces 0-24 h | — | — | 3075 ± 748 | — | — | 18.90 ± 1.18 |

| | | | |
|---|---|---|---|
| Sum total of | 98.17 ± 3.26 | 103.3 ± 3.77 | 81.02 ± 1.35 |
| all the | | | |
| organs*** | | | |
| Balance*** | — | — | 101.6 ± 2.06 |

*Blood samples are contained in the remainder of the body
**58 ml of blood/kg of KGW
***Balance without blood values, which the latter contain in the remainder of the body
****Only tissue aliquot

### EXAMPLE 39

### Organ Distribution (Including Lymph Node Concentration) After Intravenous Administration of the Contrast Medium in Prostate-Cancer-Carrying Rats

After intravenous administration of 200 μmol of total gadolinium/kg of KGW of a galenical formulation that consists of Gd-GlyMe-DOTA-trimer-perfluorooctyl-oxadecylamide (complex III) and mannose-perfluorooctylsulfonamide (Example 11; proportion 40 mol %) in rats (Cop-inbreeding Dunning R3327 MAT-Lu prostate cancer i.m.—implanted 12 days earlier), the metal content in various organs as well as in the lymph nodes (pooled as mesenteric and peripheral lymph nodes) was determined 10 minutes, 1 and 24 hours after administration (MW±SD, n=3).

| | Complex I + Substance of Example 11 | | | | | |
|---|---|---|---|---|---|---|
| | Gd Concentration | | | % Dose | | |
| | [μmol/l] | | | per total tissue | | |
| | 10 min | 1 h | 24 h | 10 min | 1 h | 24 h |
| | p.i. | p.i. | p.i. | p.i. | p.i. | p.i. |
| Liver | 183 ± 18 | 135 ± 12 | 61 ± 7 | 2.80 ± 0.20 | 2.09 ± 0.31 | 1.04 ± 0.01 |
| Spleen | 165 ± 12 | 133 ± 10 | 58 ± 7 | 0.12 ± 0.00 | 0.10 ± 0.01 | 0.04 ± 0.01 |
| Pancreas | 249 ± 85 | 161 ± 11 | 25 ± 6 | 0.33 ± 0.11 | 0.22 ± 0.04 | 0.03 ± 0.00 |
| Kidney | 2072 ± 214 | 1535 ± 654 | 378 ± 54 | 5.61 ± 0.58 | 4.23 ± 2.02 | 1.02 ± 0.13 |
| Lung | 510 ± 25 | 391 ± 40 | 44 ± 2 | 1.05 ± 0.11 | 0.78 ± 0.11 | 0.09 ± 0.01 |
| Heart | 274 ± 25 | 262 ± 43 | 17 ± 2 | 0.32 ± 0.04 | 0.35 ± 0.11 | 0.02 ± 0.00 |
| Brain | 33 ± 3 | 22 ± 2 | 2 ± 0 | 0.11 ± 0.01 | 0.07 ± 0.00 | 0.01 ± 0.00 |
| Muscle**** | 70 ± 1 | 51 ± 5 | 5 ± 2 | 0.07 ± 0.01 | 0.06 ± 0.02 | 0.00 ± 0.00 |
| Tumor | 194 ± 19 | 261 ± 12 | 47 ± 10 | 0.19 ± 0.03 | 0.24 ± 0.03 | 0.03 ± 0.01 |
| Femur | 125 ± 8 | 104 ± 10 | 11 ± 2 | 0.46 ± 0.04 | 0.41 ± 0.06 | 0.04 ± 0.00 |
| Mes. LK | 266 ± 18 | 167 ± 4 | 52 ± 5 | 0.10 ± 0.01 | 0.06 ± 0.01 | 0.02 ± 0.00 |
| Periph. LK | 260 ± 10 | 277 ± 32 | 43 ± 4 | 0.16 ± 0.02 | 0.20 ± 0.02 | 0.03 ± 0.00 |
| Stomach | 191 ± 25 | 143 ± 12 | 14 ± 3 | 0.49 ± 0.06 | 0.37 ± 0.03 | 0.04 ± 0.01 |
| (emptied) | | | | | | |
| Intestine | 200 ± 8 | 147 ± 4 | 25 ± 1 | 1.52 ± 0.02 | 1.14 ± 0.06 | 0.02 ± 0.01 |
| (emptied) | | | | | | |
| (Blood)* | 1039 ± 34 | 709 ± 28 | 17 ± 2 | 30.09 ± 0.81 | 20.51 ± 0.64 | 0.49 ± 0.07 |
| Remainder of | 427 ± 10 | 428 ± 21 | 36 ± 4 | 67.77 ± 2.28 | 66.57 ± 4.46 | 5.77 ± 0.56 |
| the body | | | | | | |
| Urine 0-24 h | — | — | 509 ± 93 | — | — | 81.34 ± 1.90 |
| Feces 0-24 h | — | — | 669 ± 224 | — | — | 8.33 ± 2.62 |

| | | | |
|---|---|---|---|
| Sum total of | 80.42 ± 2.42 | 76.43 ± 4.43 | 8.31 ± 0.73 |
| all the | | | |
| organs*** | | | |
| Balance*** | — | — | 97.98 ± 3.27 |

*Blood samples are contained in the remainder of the body
**58 ml of blood/kg of KGW
***Balance without blood values, which the latter contain in the remainder of the body
****Only tissue aliquot

Without further elaboration, it is believed that one skilled in the art can, using the preceding description, utilize the present invention to its fullest extent. The above preferred specific embodiments are, therefore, to be construed as merely illustrative, and not limitative of the remainder of the disclosure in any way whatsoever.

In the foregoing and in the above examples, all temperatures are set forth uncorrected in degrees Celsius; and, unless otherwise indicated, all parts and percentages are by weight.

The entire disclosures of all applications, patents and publications, cited above, and of corresponding German application No. 199 48 651.4-43, filed Sep. 29, 1999, and U.S. Provisional Application Ser. No. 60/158,302, filed Oct. 8, 1999, are hereby incorporated by reference.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Galenical formulations, characterized in that it contains paramagnetic and diamagentic perfluoroalkyl-containing substances.

2-38. (canceled)
